SEQUENCE LISTING (20 sequences)

SEQ ID NO: 1 - DNA, 11 bp, synthetic construct
  source 1..11: mol_type = other DNA
cgaagggcatg

SEQ ID NO: 2 - DNA, 25 bp, synthetic construct
  source 1..25: mol_type = other DNA
ctcatcatgcagctcatgcccttcg

SEQ ID NO: 3 - DNA, 14 bp, synthetic construct
  source 1..14: mol_type = other DNA
agctaaataatgag

SEQ ID NO: 4 - DNA, 33 bp, synthetic construct
  source 1..33: mol_type = other DNA
agctcatcatgcagctcatgcccttcgtaggac

SEQ ID NO: 5 - DNA, 15 bp, synthetic construct
  source 1..15: mol_type = other DNA
gcctcattttgtgct

SEQ ID NO: 6 - skipped

SEQ ID NO: 7 - DNA, 11 bp, synthetic construct
  source 1..11: mol_type = other DNA
cgccaatattt

SEQ ID NO: 8 - skipped

SEQ ID NO: 9 - DNA, 28 bp, synthetic construct
  source 1..28: mol_type = other DNA
ttcccgtcgctatcaagacatctaggac

SEQ ID NO: 10 - DNA, 11 bp, synthetic construct
  source 1..11: mol_type = other DNA
agcgacgggaa

SEQ ID NO: 11-16 - skipped (6 entries)

SEQ ID NO: 17 - DNA, 25 bp, synthetic construct
  source 1..25: mol_type = other DNA
ctcatcacgcagctcatgcccttcg

SEQ ID NO: 18 - DNA, 26 bp, synthetic construct
  source 1..26: mol_type = other DNA
gtcaagatcacagattttgggcgggc

SEQ ID NO: 19 - DNA, 26 bp, synthetic construct
  source 1..26: mol_type = other DNA
gtcaagatcacagattttgggctggc

SEQ ID NO: 20 - DNA, 26 bp, synthetic construct
  source 1..26: mol_type = other DNA
tagtataaaagcagacattttatgca

(19) United States
(12) Patent Application Publication
(10) Pub. No.: US 2024/0036035 A1
(43) Pub. Date: Feb. 1, 2024

(54) ANALYTE DETECTION USING FLUOROGENIC PROBES OR MULTIPLEX TECHNOLOGIES

(71) Applicant: The Regents of the University of Michigan, Ann Arbor, MI (US)

(72) Inventors: Nils G. Walter, Ann Arbor, MI (US); Shankar Mandal, Ann Arbor, MI (US); Aaron T. Blanchard, Ann Arbor, MI (US)

(21) Appl. No.: 18/351,263

(22) Filed: Jul. 12, 2023

Related U.S. Application Data
(60) Provisional application No. 63/399,544, filed on Aug. 19, 2022.
(60) Provisional application No. 63/389,430, filed on Jul. 15, 2022.

(51) Int. Cl. G01N 33/542; G01N 33/557

(52) U.S. Cl. CPC G01N 33/542; G01N 33/557

(57) ABSTRACT

Provided herein is technology relating to detecting analytes and particularly, but not exclusively, to methods, compositions, systems, and kits for detecting analytes using fluorogenic probes and multiplex technologies.

This application claims priority to U.S. provisional patent application Ser. No. 63/389,430, filed Jul. 15, 2022, and U.S. provisional patent application Ser. No. 63/399,544, filed Aug. 19, 2022, each of which is incorporated herein by reference in its entirety.

### STATEMENT REGARDING FEDERALLY SPONSORED RESEARCH OR DEVELOPMENT

This invention was made with government support under CA229023 and CA245789 awarded by the National Institutes of Health. The government has certain rights in the invention.

### SEQUENCE LISTING

The text of the computer readable sequence listing filed herewith, titled “UM-40922-203_SQL”, created Jul. 12, 2023, having a file size of 13,228 bytes, is hereby incorporated by reference in its entirety.

### FIELD

Provided herein is technology relating to detecting analytes and particularly, but not exclusively, to methods, compositions, systems, and kits for detecting analytes using fluorogenic probes and/or multiplex technologies.

### BACKGROUND

Sensitive and accurate detection, quantification, identification, and/or characterization of biomarkers finds use in clinical diagnostics for differentiating between healthy and diseased states. Accordingly, diagnosis and treatment of disease would benefit from new technologies for rapid and accurate analysis of biomarkers.

### SUMMARY

Single Molecule Recognition through Equilibrium Poisson Sampling (SiMREPS) has emerged as a powerful technique for the ultrasensitive and specific detection of protein and other biomarkers, with LODs in the aM to low fM range (see, e.g., U.S. Pat. No. 10,093,967; U.S. Pat. App. Pub. Nos. 2021/0348230; 2021/0230688; 2021/0292837; 2018/0258469; 2019/0187031; 2021/0318296; and U.S. Pat. App. Ser. No. 63/224,984, each of which is incorporated herein by reference. The high sensitivity of SiMREPS results from use of binding and dissociation kinetics to distinguish signals of specific binding of query probes to an analyte from nonspecific binding (e.g., to assay surfaces or matrix) or binding to non-target analytes. In particular, previous SiMREPS technologies have used nucleic acid or antibody query probes with relatively fast dissociation kinetics (k_{off} of approximately 0.05-0.5 s⁻¹). The query probes repeatedly associate and dissociate with target analytes, which provides a repeated interrogation of single target molecules and generates characteristic kinetic fingerprints within a reasonably short acquisition time (e.g., 2 min per field of view) without sacrificing sensitivity.

Accordingly, provided herein is a method for detecting an analyte. For example, in some embodiments, the method comprises stably binding an analyte to a solid support; providing an analyte-specific fluorogenic query probe comprising a detectable label and a quencher of the detectable label; and recording a time-dependent change in a signal intensity of the detectable label. In some embodiments, the solid support comprises an immobilized capture probe and stably binding the analyte to the solid support comprises stably binding the analyte to the immobilized capture probe. In some embodiments, the detectable label comprises a fluorescent moiety and the quencher comprises a quenching moiety. In some embodiments, the solid support is diffusible. In some embodiments, the analyte comprises a nucleic acid. In some embodiments, the capture probe comprises an antibody or antigen-binding antibody fragment. In some embodiments, the capture probe comprises a nucleic acid. In some embodiments, transient association of the query probe with the analyte produces the time-dependent change in the signal intensity of the detectable label. In some embodiments, the method further comprises counting a number of changes in the signal intensity of the detectable label. In some embodiments, the method further comprises determining a value for N_{b+d}. In some embodiments, the method further comprises determining a value for τ_{on, median}. In some embodiments, the method further comprises providing a sample comprising the analyte. In some embodiments, the analyte is present at a concentration of approximately 100 fM. In some embodiments, the analyte is present at a concentration of 10 to 1000 fM. In some embodiments, the sample is a biological sample. In some embodiments, stably binding the analyte to the solid support comprises contacting the sample to the solid support.

In some embodiments, the fluorogenic probe comprises 13 to 17 nucleotides (e.g., 13, 14, 15, 16, or 17 nucleotides). In some embodiments, the fluorogenic probe comprises a nucleotide sequence comprising 3 to 7 mismatched bases (e.g., 3, 4, 5, 6, or 7 mismatched bases) with respect to the nucleotide sequence of the analyte. In some embodiments, the fluorogenic probe is secondary structure-free.

In some embodiments, the nucleotide sequence of the fluorogenic probe is identified using an iterative optimization process comprising providing an initial query probe nucleotide sequence of approximately 15 nt (e.g., 13, 14, 15, 16, or 17 nt) that is fully complementary to a target nucleic acid analyte; producing a candidate probe nucleotide sequence from the initial query probe nucleotide sequence by selecting approximately 3 to 7 nucleotides (e.g., 3, 4, 5, 6, or 7 nucleotides) and changing the selected nucleotides to a different nucleotide base; calculating the free energy of association for the candidate probe nucleotide sequence with the target analyte sequence; calculating the free energy of intermolecular secondary structures and/or self-dimers formed by the candidate probe nucleotide sequence; and identifying a candidate probe nucleotide sequence comprising a nucleotide sequence that maximizes specificity for the target nucleic acid analyte and that minimizes tendency to form secondary structures and self-dimers to be used as a fluorogenic probe. In some embodiments, the method comprises producing at least 1000 candidate probe nucleotide sequences.

In some embodiments, the fluorogenic query probe detectable label and quencher form a ground-state stabilization complex when the fluorogenic query probe is not associated with a target analyte. In some embodiments, recording the time-dependent change in the signal intensity of the detectable label comprises recording a series of images. In some embodiments, the method comprises producing an intensity fluctuation map by determining an average absolute image-to-image change in intensity at a number of image pixels. In some embodiments, the method further comprises generating intensity-versus-time data and calculating a kinetic parameter from the intensity-versus-time data. In some embodiments, the method further comprises identifying positive detection events using a threshold for the kinetic parameter.

The technology further provides embodiments of systems for detecting an analyte. For example, in some embodiments, systems comprise a solid support; an analyte-specific fluorogenic query probe comprising a detectable label and a quencher of the detectable label; a detector configured to detect the detectable label; a memory configured to record time-dependent changes in a signal intensity of the detectable label; and a processor configured to generate intensity-versus-time data from the time-dependent changes in a signal intensity of the detectable label. In some embodiments, systems further comprise an analyte. In some embodiments, the analyte is stably bound to the solid support. In some embodiments, the solid support comprises a capture probe. In some embodiments, the detectable label comprise a fluorescent moiety and the quencher comprises a quenching moiety. In some embodiments, the solid support is diffusible. In some embodiments, the analyte comprises a nucleic acid. In some embodiments, the capture probe comprises an antibody or antigen-binding antibody fragment. In some embodiments, the capture probe comprises a nucleic acid. In some embodiments, transient association of the query probe with the analyte produces the time-dependent change in the signal intensity of the detectable label. In some embodiments, the processor is further configured to count a number of changes in the signal intensity of the detectable label. In some embodiments, the processor is further configured to determine a value for N_{b+d}. In some embodiments, the processor is further configured to determine a value for τ_{on, median}. In some embodiments, the analyte is present at a concentration of 100 fM. In some embodiments, the analyte is present at a concentration of 10 to 1000 fM. In some embodiments, the processor is configured to record a series of images. In some embodiments, the processor is configured to produce an intensity fluctuation map by determining an average absolute image-to-image change in intensity at a number of image pixels. In some embodiments, the processor is configured to calculate a kinetic parameter from the intensity-versus-time data. In some embodiments, the processor is configured to identify positive detection events using a threshold for the kinetic parameter.

The technology also provides use of an analyte-specific fluorogenic query probe to characterize, identify, quantify, and/or detect an analyte in a SiMREPS assay method. In some embodiments, the SiMREPS assay method comprises stably binding the analyte to a solid support; providing the analyte-specific fluorogenic query probe comprising a detectable label and a quencher of the detectable label; and recording a time-dependent change in a signal intensity of the detectable label. In some embodiments, the solid support comprises an immobilized capture probe and stably binding the analyte to the solid support comprises stably binding the analyte to the immobilized capture probe. In some embodiments, the detectable label comprise a fluorescent moiety and the quencher comprises a quencher moiety. In some embodiments, the solid support is diffusible. In some embodiments, the analyte comprises a nucleic acid. In some embodiments, the capture probe comprises an antibody or antigen-binding antibody fragment. In some embodiments, the capture probe comprises a nucleic acid. In some embodiments, transient association of the query probe with the analyte produces the time-dependent change in the signal intensity of the detectable label. In some embodiments, the SiMREPS assay method further comprises counting a number of changes in the signal intensity of the detectable label. In some embodiments, the SiMREPS assay method further comprises determining a value for N_{b+d}. In some embodiments, the SiMREPS assay method further comprises determining a value for τ_{on, median}. In some embodiments, the use further comprises providing a sample comprising the analyte. In some embodiments, the sample is a biological sample. In some embodiments, stably binding the analyte to the solid support comprises contacting the sample to the solid support. In some embodiments, the analyte is present at a concentration of approximately 100 fM. In some embodiments, the analyte is present at a concentration of 10 to 1000 fM. In some embodiments, recording the time-dependent change in the signal intensity of the detectable label comprises recording a series of images. In some embodiments, the SiMREPS assay method further comprises producing an intensity fluctuation map by determining an average absolute image-to-image change in intensity at a number of image pixels. In some embodiments, the SiMREPS assay method further comprises generating intensity-versus-time data and calculating a kinetic parameter from the intensity-versus-time data. In some embodiments, the SiMREPS assay method further comprises identifying positive detection events using a threshold for the kinetic parameter.

The technology also provides embodiments of a multiplex method for detecting a plurality of analytes. For example, in some embodiments, the method comprises stably binding a first analyte to a solid support; stably binding a second analyte to the solid support; providing a first query probe comprising a first detectable label, wherein the first query probe is specific for the first analyte; providing a second query probe comprising a second detectable label, wherein the second query probe is specific for the second analyte; recording a first time-dependent change in a first signal intensity of the first detectable label; and recording a second time-dependent change in a second signal intensity of the second detectable label. In some embodiments, the first detectable label and the second detectable label are the same. In some embodiments, the first detectable label and the second detectable label are different. In some embodiments, the first analyte comprises a nucleic acid, a protein, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism. In some embodiments, the second analyte comprises a nucleic acid, a protein, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism. In some embodiments, the method comprises using the first time-dependent change in the first signal intensity to characterize, identify, quantify, and/or detect the first analyte and using the second time-dependent change in the second signal intensity to characterize, identify, quantify, and/or detect the second analyte. In some embodiments, the first time-dependent change in the first signal intensity and the second time-dependent change in the second signal intensity are recorded in a single field of view. In some embodiments, the method comprises using Poisson statistical treatment to distinguish the first time-dependent change in the first signal intensity and the second time-dependent change in the second signal intensity. In some embodiments, the first time-dependent change in the first signal intensity and the second time-dependent change in the second signal intensity are distinguishable by a difference in one or more of: signal intensity, dwell time in the unbound state, dwell time in the bound state, kinetic dissociation constant, and/or kinetic association constant.

The technology provides embodiments of a system for detecting an analyte. For example, in some embodiments, the system comprises a solid support; a first query probe comprising a first detectable label, wherein the first query probe is specific for the first analyte; a second query probe comprising a second detectable label, wherein the second query probe is specific for the second analyte; a detector configured to detect the first detectable label and to detect the second detectable label; a memory configured to record first time-dependent changes in a first signal intensity of the first detectable label and to record second time-dependent changes in a second signal intensity of the second detectable label; and a processor configured to generate first intensity-versus-time data from the first time-dependent changes in the first signal intensity of the first detectable label and to generate second intensity-versus-time data from the second time-dependent changes in the second signal intensity of the second detectable label. In some embodiments, the system further comprises a first analyte and a second analyte. In some embodiments, the first analyte is stably bound to the solid support and the second analyte is stably bound to the solid support. In some embodiments, the solid support comprises a first capture probe and a second capture probe. In some embodiments, the first detectable label comprise a first fluorescent moiety and the second detectable label comprises a second fluorescent moiety. In some embodiments, the first detectable label and the second detectable label are different. In some embodiments, the first detectable label and the second detectable label are the same. In some embodiments, the solid support is diffusible. In some embodiments, the first analyte comprises a nucleic acid, a protein, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism; and the second analyte comprises a nucleic acid, a protein, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism. In some embodiments, the first capture probe comprises an antibody or antigen-binding antibody fragment or a nucleic acid. In some embodiments, the second capture probe comprises an antibody or antigen-binding antibody fragment or a nucleic acid. In some embodiments, transient association of the first query probe with the first analyte produces the first time-dependent change in the first signal intensity of the first detectable label and transient association of the second query probe with the second analyte produces the second time-dependent change in the second signal intensity of the second detectable label. In some embodiments, the processor is further configured to count a first number of changes in the first signal intensity of the first detectable label and to count a second number of changes in the second signal intensity of the second detectable label. In some embodiments, the processor is further configured to determine a first value for N_{b+d} for a first analyte and to determine a second value for N_{b+d} for a second analyte. In some embodiments, the processor is further configured to determine a first value for τ_{on, median} for a first analyte and to determine a second value for τ_{on, median} for a second analyte. In some embodiments, the processor is configured to record a series of images. In some embodiments, the processor is configured to produce an intensity fluctuation map by determining an average absolute image-to-image change in intensity at a number of image pixels. In some embodiments, the processor is configured to calculate a first kinetic parameter from the intensity-versus-time data and to calculate a second kinetic parameter from the intensity-versus-time data. In some embodiments, the processor is configured to identify positive detection events using a threshold for the first and second kinetic parameters. While the multiplex technology is described herein in reference to a first and second analyte, the technology also comprises a multiplex technology for more than two analytes, e.g., a third, fourth, fifth, sixth, . . . nth analyte. Accordingly, reference to first and second elements (e.g., first and second query probe, first and second detectable label, first and second intensity-versus-time data, first and second time-dependent changes, first and second signal intensity, first and second query probe, etc.) be extended to include a third, fourth, fifth, sixth, . . . nth element to provide the multiplex technology.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A shows the sequences of an exemplary (T790M) target analyte, capture probe, and fluorogenic query probe “F-gen V4”. The F-gen V4 has 14 nucleotides and contains 3 nucleotides that were mismatched to the T790M target analyte (Table 1). Arrows in FIG. 1A indicate the positions of mismatches between the query probe and the target analyte.

FIG. 1B-D show data collected from fluorogenic probe SiMREPS experiments in which a sample comprising 100 fM of the T790M target analyte was analyzed with 500 nM of the F-gen V4 query probe. Video data were collected for 10 minutes. FIG. 1B is a histogram of accepted N_{b+d} kinetic data. FIG. 1C is a plot of τ_{on, median} as a function of N_{b+d} and FIG. 1D is a plot of τ_{off, median} as a function of N_{b+d}. Representative kinetic traces are shown in FIG. 1E.

FIG. 2A-2C show data collected from fluorogenic probe SiMREPS experiments in which a sample comprising 100 fM of the T790M target analyte in 10% w/v formamide was analyzed with 500 nM of the F-gen V4. Video data were collected for 10 minutes. FIG. 2A is a histogram of accepted N_{b+d} kinetic data. FIG. 2B is a plot of τ_{on, median} as a function of N_{b+d} and FIG. 2C is a plot of τ_{off, median} as a function of N_{b+d}. Representative kinetic traces for the first 30 seconds of the video data are shown in FIG. 2D.

FIG. 3A-3C show data from control experiments in which a sample with no target was analyzed with 500 nM of the F-gen V4 query probe. No traces were acceptable in the control experiment. FIG. 3A is a histogram of accepted N_{b+d} kinetic data (none accepted). FIG. 3B is a plot of τ_{off, median} as a function of N_{b+d} and FIG. 3C is a plot of τ_{on, median} as a function of N_{b+d}.

FIG. 4A-4F show data from experiments conducted to test use of a fluorogenic query probe to detect a target analyte. FIG. 4A-4C show data collected from experiments in which a sample comprising 100 fM of the target analyte in IB 10% w/v formamide was analyzed with 500 nM of the F-gen V4 query probe. FIG. 4A, FIG. 4B, and FIG. 4C show experimental data collected from 2.5, 5, or 10 minutes of video, respectively. FIG. 4D, FIG. 4E, and FIG. 4F show data collected from 2.5, 5, or 10 minutes of video, respectively, from control experiments performed under the same conditions except samples were prepared without the target analyte. The number of accepted traces obtained from each analysis were comparable for the three different data collection times. One false positive was detected in the control experiment data (FIG. 4D-4F).

FIG. 5A-5C show the results of experiments comparing conventional SiMREPS (FIG. 5A), an intramolecular SiMREPS technology (FIG. 5B), and the fluorogenic probe SiMREPS as described herein (FIG. 5C).

FIG. 6A is a schematic drawing showing a multiplex SiMREPS assay design.

FIG. 6B is a schematic drawing showing a multiplex SiMREPS assay design.

FIG. 7A is a schematic drawing showing capture probes used in multiplex SiMREPS experiments described herein.

FIG. 7B is a schematic drawing showing target analytes captured by capture probes and query probes used in multiplex SiMREPS experiments described herein.

FIG. 7C shows data from separate non-multiplex SiMREPS experiments for detecting miR-16 and IL-6 as depicted in FIG. 7A and FIG. 7B. These data were used to train models used to identify miR-16 and IL-6 in multiplex SiMREPS.

FIG. 7D shows data from multiplex SiMREPS for detecting miR-16 and IL-6 as depicted in FIG. 7A and FIG. 7B.

FIG. 8A is a schematic drawing showing target analytes captured by capture probes and query probes used in multiplex SiMREPS experiments described herein.

FIG. 8B shows kinetic data collected in SiMREPS experiments using query probes for detecting EGFR exon 19 deletion, IL-6, and miR-16 as depicted in FIG. 8A.

FIG. 8C shows plots of τ_{on, median} versus N_{b+d} for the data shown in FIG. 8B.

FIG. 9A shows exemplary kinetic data for five different analytes that may be differentiated by shapes of the intensity-versus-time plots.

FIG. 9B shows an exemplary multiplex technology in which analytes are distinguished using different colors of fluorescent moieties on query probes and by different shapes of the intensity-versus-time plots.

FIG. 10 shows a bar graph indicating the number of single-molecule traces that were accepted as positive detection events for the target proteins beta-NGF, IL-10, or IL-11 in a 3-plex SiMREPS measurement in which all three targets were distinguished based on their characteristic kinetic fingerprints. In this measurement, all three target proteins were detected simultaneously in a single 5-minute acquisition per sample well, using a single type of fluorophore (Alexa Fluor 647) and a single excitation wavelength (640 nanometers). In the table below the graph, ‘+’ indicates that a given target was present in the sample and ‘−’ indicates that the target was absent.

FIG. 11 shows a bar graph indicating the number of single-molecule traces that were accepted as positive detection events for the target proteins IL-11, IL-8, or IL-6 in a 3-plex SiMREPS measurement in which all three targets were distinguished based on their characteristic kinetic fingerprints. In this measurement, all three target proteins were detected simultaneously in a single 5-minute acquisition per sample well, using a single type of fluorophore (Alexa Fluor 647) and a single excitation wavelength (640 nanometers). In the table below the graph, ‘+’ indicates that a given target was present in the sample, and ‘−’ indicates that the target was absent.

FIG. 12A shows a schematic of the experimental design for the fluorogenic SiMREPS (FG-SiMREPS) experiments.

FIG. 12B provides sequences of nucleic acid biomarker targets analyzed during the experiments: Exon20 T790M (SEQ ID NO: 12), Exon29 T790 WT (SEQ ID NO: 13), EGFR L858R (SEQ ID NO: 14), EGFR L858 WT (SEQ ID NO: 15), and HPV16 (SEQ ID NO: 16).

FIG. 12C shows plots of raw intensity-time trajectories and hidden Markov model (HMM) trajectories.

FIG. 12D shows distributions of the number of binding and dissociation events (N_{b+d}) for the data shown is FIG. 12C.

FIG. 13A shows a schematic of multiplexed SiMREPS using kinetic fingerprinting and different colors.

FIG. 13B shows data from experiments conducted to identify experimental and imaging conditions for use in detecting six different microRNAs (miRNAs).

FIG. 14A shows data collected from kinetic and optical multiplexed SiMREPS detection of miRNA.

FIG. 14B shows the optical multiplexing of three miRNAs.

FIG. 15A shows ratio-metric multiplexing to detect two miRNAs.

FIG. 15B shows concentration estimation from accepted counts obtained per FOV from the same multiplexing assay as in FIG. 15A.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detecting analytes and particularly, but not exclusively, to methods, compositions, systems, and kits for detecting analytes using fluorogenic probes and/or multiplex technologies.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., an analyte). For example, when an analyte is said to be “present” in a test sample, it means the level or amount of this analyte is above a pre-determined threshold; conversely, when an analyte is said to be “absent” in a test sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Another relative change indicating an “increase” or “decrease” is a change in a measured value that is at least 2 or 3 times the standard deviation of background noise. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

As used herein, the terms “subject” and “patient” refer to any organisms including plants, microorganisms, and animals (e.g., mammals such as dogs, cats, livestock, and humans).

The term “sample” in the present specification and claims is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

The term “label” as used herein refers to any atom, molecule, molecular complex (e.g., metal chelate), or colloidal particle (e.g., quantum dot, nanoparticle, microparticle, etc.) that can be used to provide a detectable (preferably quantifiable) effect, and that can be attached to a nucleic acid or protein. Labels include, but are not limited to, dyes (e.g., optically-detectable labels, fluorescent dyes or moieties, etc.); radiolabels such as ³²P; binding moieties such as biotin; haptens such as digoxigenin; luminogenic, phosphorescent, optically-detectable, or fluorogenic moieties; mass tags; and fluorescent dyes alone or in combination with moieties that can suppress or shift emission spectra by fluorescence resonance energy transfer (FRET). Labels may provide signals detectable by fluorescence, luminescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein, the term “support” or “solid support” refers to a matrix on or in which an analyte, capture probe, and the like may be immobilized, e.g., to which they may be covalently or noncovalently attached or in or on which they may be partially or completely embedded so that they are largely or entirely prevented from diffusing freely or moving with respect to one another.

As used herein, “moiety” refers to one of two or more parts into which something may be divided, such as, for example, the various parts of an oligonucleotide, a molecule, a chemical group, a domain, a probe, a polypeptide, etc.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the terms “complementary” or “complementarity” are used in reference to polynucleotides (e.g., a sequence of nucleotides such as an oligonucleotide capture probe, query probe or a target analyte that is a nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, the term “mismatch” means a nucleobase of a first nucleic acid that is not capable of pairing with a nucleobase at a corresponding position of a second nucleic acid.

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include transmembrane domains, glycosylation sites, etc.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; in some embodiments, one sequence is a reference sequence.

The term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46461 (1960) have been followed by the refinement of this process into an essential tool of modern biology.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see, e.g., Anderson and Young, Quantitative Filter Hybridization, in Nucleic Acid Hybridization (1985). Other references (e.g., Allawi and SantaLucia, Biochemistry 36: 10581-94 (1997) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine Val, V. As used herein, the codes Xaa and X refer to any amino acid.

The terms “variant” and “mutant” when used in reference to a polypeptide refer to an amino acid sequence that differs by one or more amino acids from another, usually related polypeptide.

As used herein, the term “melting” when used in reference to a nucleic acid refers to the dissociation of a double-stranded nucleic acid or region of a nucleic acid into a single-stranded nucleic acid or region of a nucleic acid.

As used herein, a “query probe” or “reader probe” is any entity (e.g., molecule, biomolecule, etc.) that recognizes an analyte (e.g., binds to an analyte, e.g., binds specifically to an analyte). In exemplary embodiments, the query probe is a protein that recognizes an analyte. In some other exemplary embodiments, the query probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, the query probe is labeled, e.g., with a detectable label such as, e.g., a fluorescent moiety as described herein. In some embodiments, the query probe comprises more than one type of molecule (e.g., more than one of a protein, a nucleic acid, a chemical linker or a chemical moiety).

As used herein, an “event” refers to an instance of a query probe binding to an analyte or an instance of query probe dissociation from an analyte, e.g., as measured by monitoring a detectable property indicating the binding of a query probe to an analyte and/or the dissociation of a query probe from an analyte.

As used herein, the term “N_{b+d}” refers to the number of binding (b) and dissociation (d) events of one or more detectably labeled query probes observed at a single location (e.g., a discrete region of a solid support, a single diffraction-limited region of an image, or a single location of a specimen as determined by super-resolution imaging) within a defined time window of observation (e.g., 10 seconds, 20 seconds, 30 seconds, 1 minute, . . . , etc.) N_{b+d} is determined by counting the number of sudden (e.g., within a span of time smaller than the time resolution of the measurement, e.g., less than approximately 500 milliseconds or less than approximately 100 milliseconds) increases (corresponding to binding events) and decreases (corresponding to dissociation events) in query probe signal (e.g., fluorescence intensity) at a single location within the observation window. The counting of binding and dissociation events may be performed using one or more of several different algorithms, including but not limited to: edge detection algorithms, hidden Markov models, super-resolution analysis of intensity difference maps, k-means clustering, least-squares fitting, machine learning, or deep learning.

As used herein, the term τ_{on, median} (also known as τ_{bound, median}) refers to the median value of the apparent residence times of all detectably labeled query probes observed at a single location (e.g., a discrete region of a solid support, a single diffraction-limited region of an image, or a single location of a specimen as determined by super-resolution imaging) within a defined time window of observation (e.g., 10 seconds, 20 seconds, 30 seconds, 1 minute, . . . , etc.) As used herein, a “capture probe” is any entity (e.g., molecule, biomolecule, etc.) that recognizes an analyte (e.g., binds to an analyte, e.g., binds specifically to an analyte) and links the analyte to a solid support. In exemplary embodiments, the capture probe is a protein that recognizes an analyte. In some other exemplary embodiments, a capture probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, a capture probe is labeled, e.g., with a detectable label such as, e.g., a fluorescent moiety as described herein. In some embodiments, the capture probe comprises more than one type of molecule (e.g., more than one of a protein, a nucleic acid, a chemical linker or a chemical moiety).

As used herein, the term “sensitivity” refers to the probability that an assay gives a positive result for the analyte when the sample comprises the analyte. Sensitivity is calculated as the number of true positive results divided by the sum of the true positives and false negatives. Sensitivity is a measure of how well an assay detects an analyte.

As used herein, the term “specificity” refers to the probability that an assay gives a negative result when the sample does not comprise the analyte. Specificity is calculated as the number of true negative results divided by the sum of the true negatives and false positives. Specificity is a measure of how well a method of the present invention excludes samples that do not comprise an analyte from those that do comprise the analyte.

As used herein, the “equilibrium constant” (K_{eq}), the “equilibrium association constant” (Kₐ), and “association binding constant” (or “binding constant” (K_{B})) are used interchangeably for the following binding reaction of A and B at equilibrium:

A+B [symbol] AB

where A and B are two entities that associate with each other (e.g., capture probe and analyte, query probe and analyte) and K_{eq}=[AB]/([A]×[B]). The dissociation constant K_{D}=1/K_{B}. The K_{D} is a useful way to describe the affinity of a one binding partner A for a partner B with which it associates, e.g., the number K_{D} represents the concentration of A or B that is required to yield a significant amount of AB. K_{eq}=k_{off}/kₒₙ; K_{D}=k_{off}/kₒₙ.

As used herein, a “significant amount” of the product of two entities that associate with each other, e.g., formation of AB from A and B according to the equation above, refers to a concentration of AB that is equal to or greater than the free concentration of A or B, whichever is smaller.

As used herein, “nanomolar affinity range” refers to the association of two components that has an equilibrium dissociation constant K_{D} (e.g., ratio of k_{off}/kₒₙ) in the nanomolar range, e.g., a dissociation constant (K_{D}) of 1×10⁻¹⁰ to 1×10⁻⁵ M (e.g., in some embodiments 1×10⁻⁹ to 1×10⁻⁶ M). The dissociation constant has molar units (M). The smaller the dissociation constant, the higher the affinity between two components (e.g., capture probe and analyte; query probe and analyte).

As used herein, a “weak affinity” or “weak binding” or “weak association” refers to an association having a K_{D} of approximately 100 nanomolar (e.g., approximately 10, 20, 30, 50, 60, 70, 80, 90, 100, 110, 120, 130, 140, 150, 160, 170, 180, 190, 200, 300, 400, or 500 nanomolar) and/or, in some embodiments, in the range of 1 nanomolar to 10 micromolar.

The terms “specific binding” or “specifically binding” when used in reference to the interaction of two components A and B that associate with one another refers to an association of A and B having a K_{D} that is smaller than the K_{D} for the interaction of A or B with other similar components in the solution, e.g., at least one other molecular species in the solution that is not A or B.

The term “detection assay” refers to an assay for detecting the presence or absence of an analyte or the activity or effect of an analyte or for detecting the presence or absence of a variant of an analyte.

In some embodiments the technology comprises an antibody component or moiety, e.g., an antibody or fragments or derivatives thereof. As used herein, an “antibody”, also known as an “immunoglobulin” (e.g., IgG, IgM, IgA, IgD, IgE), comprises two heavy chains linked to each other by disulfide bonds and two light chains, each of which is linked to a heavy chain by a disulfide bond. The specificity of an antibody resides in the structural complementarity between the antigen combining site of the antibody (or paratope) and the antigen determinant (or epitope). Antigen combining sites are made up of residues that are primarily from the hypervariable or complementarity determining regions (CDRs). Occasionally, residues from non-hypervariable or framework regions influence the overall domain structure and hence the combining site. Some embodiments comprise a fragment of an antibody, e.g., any protein or polypeptide-containing molecule that comprises at least a portion of an immunoglobulin molecule such as to permit specific interaction between said molecule and an antigen. The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof. Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques or can be prepared as a contiguous protein using genetic engineering techniques.

Fragments of antibodies include, but are not limited to, Fab (e.g., by papain digestion), F(ab′)₂ (e.g., by pepsin digestion), Fab′ (e.g., by pepsin digestion and partial reduction) and Fv or scFv (e.g., by molecular biology techniques) fragments.

A Fab fragment can be obtained by treating an antibody with the protease papain. Also, the Fab may be produced by inserting DNA encoding a Fab of the antibody into a vector for prokaryotic expression system or for eukaryotic expression system and introducing the vector into a prokaryote or eukaryote to express the Fab. A F(ab′)₂ may be obtained by treating an antibody with the protease pepsin. Also, the F(ab′)₂ can be produced by binding a Fab′ via a thioether bond or a disulfide bond. A Fab may be obtained by treating F(ab′)₂ with a reducing agent, e.g., dithiothreitol. Also, a Fab′ can be produced by inserting DNA encoding a Fab′ fragment of the antibody into an expression vector for a prokaryote or an expression vector for a eukaryote and introducing the vector into a prokaryote or eukaryote for its expression. A Fv fragment may be produced by restricted cleavage by pepsin, e.g., at 4° C. and pH 4.0. (a method called “cold pepsin digestion”). The Fv fragment consists of the heavy chain variable domain (V_{H}) and the light chain variable domain (V_{L}) held together by strong noncovalent interaction. A scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv.

In general, antibodies can usually be raised to any antigen, using the many conventional techniques now well known in the art.

As used herein, the term “conjugated” refers to when one molecule or agent is physically or chemically coupled or adhered to another molecule or agent. Examples of conjugation include covalent linkage and electrostatic complexation. The terms “complexed,” “complexed with,” and “conjugated” are used interchangeably herein.

As used herein, a “stable interaction” or referring to a “stably bound” interaction refers to an association that is relatively persistent under the thermodynamic equilibrium conditions of the interaction. In some embodiments, a “stable interaction” is an interaction between two components having a K_{D} that is smaller than approximately 10⁻⁹ M or, in some embodiments a K_{D} that is smaller than 10⁻⁸ M. In some embodiments, a “stable interaction” has a dissociation rate constant k_{off} that is smaller than 1 per hour or, in some embodiments, a dissociation rate constant k_{off} that is smaller than 1 per minute. In some embodiments, a “stable interaction” is defined as not being a “transient interaction”. In some embodiments, a “stable interaction” includes interactions mediated by covalent bonds and other interactions that are not typically described by a K_{D} value but that involve an average association lifetime between two entities that is longer than approximately 1 minute (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 seconds) per each interaction.

In some embodiments, the distinction between a “stable interaction” and a “transient interaction” is determined by a cutoff value of K_{D} and/or k_{off} and/or another kinetic or thermodynamic value describing the associations, wherein the cutoff is used to discriminate between stable and transient interactions that might otherwise be characterized differently if described in absolute terms of a K_{D} and/or k_{off} or another kinetic or thermodynamic value describing the associations. For example, a “stable interaction” characterized by a K_{D} value might also be characterized as a “transient interaction” in the context of another interaction that is even more stable. One of skill in the art would understand other relative comparisons of stable and transient interactions, e.g., that a “transient interaction” characterized by a K_{D} value might also be characterized as a “stable interaction” in the context of another interaction that is even more transient (less stable).

As used herein, “moiety” refers to one of two or more parts into which something may be divided, such as, for example, the various parts of an oligonucleotide, a molecule, a chemical group, a domain, a probe, an “R” group, a polypeptide, etc.

As used herein, in some embodiments a “signal” is a time-varying quantity associated with one or more properties of a sample that is assayed, e.g., the binding of a query probe to an analyte and/or dissociation of a query probe from an analyte. A signal can be continuous in the time domain or discrete in the time domain. As a mathematical abstraction, the domain of a continuous-time signal is the set of real numbers (or an interval thereof) and the domain of a discrete-time signal is the set of integers (or an interval thereof). Discrete signals often arise via “digital sampling” of continuous signals. For example, an audio signal consists of a continually fluctuating voltage on a line that can be digitized by reading the voltage level on the line at a regular interval, e.g., every 50 microseconds. The resulting stream of numbers is stored as a discrete-time digital signal. In some embodiments, the signal is recorded as a function of location is space (e.g., x, y coordinates; e.g., x, y, z coordinates). In some embodiments, the signal is recorded as a function of time. In some embodiments, the signal is recorded as a function of time and location.

The term “substantially” as used herein, is a broad term and is used in its ordinary sense, including, but not limited to, being largely but not necessarily wholly that which is specified.

The term “algorithm,” as used herein, is a broad term and is used in its ordinary sense, including, but not limited to, the computational processes (for example, programs) involved in transforming information from one state to another, for example using computer processing.

### DESCRIPTION

Although the disclosure herein refers to certain illustrated embodiments, it is to be understood that these embodiments are presented by way of example and not by way of limitation.

### Poisson Processes

Embodiments of the technology are related to single-molecule recognition by recording the characteristic kinetics of a query probe binding to a target analyte. In particular embodiments, this process is a Poisson process. A Poisson process is a continuous-time stochastic process that counts the number of events and the time that events (e.g., transient binding of a detectably labeled (e.g., fluorescent) query probe to an immobilized target analyte) occur in a given time interval. The time interval between each pair of consecutive events has an exponential distribution and each interval is assumed to be independent of other intervals. The Poisson distribution is a discrete probability distribution that expresses the probability of a given number of the events occurring in the given time interval if these events occur with a known average rate and independently of the time since the last event. The Poisson distribution can also be used for the number of events in other specified intervals such as distance, area, or volume.

A Poisson distribution is a special case of the general binomial distribution where the number of trials n is large, the probability of success p is small, and the product np=λ is moderate. In a Poisson process, the probability that a number of events Nis j at any arbitrary time t follows the Poisson probability distribution Pⱼ(t):

$${P}_{j} ( t ) = \frac{{{e}^{- λ t} ( λ t )}^{j}}{j !} , j = 0 , 1 , 2 , … . \qquad ( 1 )$$

That is, the number N of events that occur up to time t has a Poisson distribution with parameter λt. Statistical and mathematical methods relevant to Poisson processes and Poisson distributions are known in the art. See, e.g., “Stochastic Processes (i): Poisson Processes and Markov Chains” in Statistics for Biology and Health—Statistical Methods in Bioinformatics (Ewans and Grant, eds.), Springer (New York, 2001), page 129 et seq., incorporated herein by reference in its entirety. Software packages such as Matlab and R may be used to perform mathematical and statistical methods associated with Poisson processes, probabilities, and distributions.
Kinetics of Detection Particular embodiments of the technology are related to detecting an analyte by analyzing the kinetics of the interaction of a query probe with the analyte to be detected. For the interaction of a query probe Q (e.g., at an equilibrium concentration [Q]) with a target analyte T (e.g., at an equilibrium concentration [T]), the kinetic rate constant kₒₙ describes the time-dependent formation of the complex QT comprising the probe Q hybridized to the analyte T. In particular embodiments, while the formation of the QT complex is associated with a second order rate constant that is dependent on the concentration of query probe and has units of M⁻¹ min⁻¹ (or the like), the formation of the QT complex is sufficiently described by a kₒₙ that is a pseudo-first order rate constant associated with the formation of the QT complex. Thus, in some embodiments, kₒₙ is an apparent (“pseudo”) first-order rate constant.

Likewise, the kinetic rate constant k_{off} describes the time-dependent dissociation of the complex QT into the probe Q and the analyte T. Kinetic rates are typically provided herein in units of min⁻¹ or s⁻¹. The “dwell time” of the query probe Q in the bound state (τₒₙ) is the time interval (e.g., length of time) that the probe Q is hybridized to the analyte T during each instance of query probe Q binding to the analyte T to form the QT complex. The “dwell time” of the query probe Q in the unbound state (τ_{off}) is the time interval (e.g., length of time) that the probe Q is not hybridized to the analyte T between each instance of query probe Q binding to the analyte to form the QT complex (e.g., the time the query probe Q is dissociated from the target analyte T between successive binding events of the query probe Q to the target analyte T). Dwell times may be provided as averages or weighted averages integrating over numerous binding and non-binding events.

Further, in some embodiments, the repeated, stochastic binding of probes (e.g., detectably labeled query probes (e.g., fluorescent probes) to target analytes is modeled as a Poisson process occurring with constant probability per unit time and in which the standard deviation in the number of binding and dissociation events per unit time (N_{b+d}) increases as (N_{b+d})^{1/2}. Thus, the statistical noise becomes a smaller fraction of N_{b+d} as the observation time is increased. Accordingly, the observation is lengthened as needed in some embodiments to achieve discrimination between target and off-target binding. And, as the acquisition time is increased, the signal and background peaks in the N_{b+d} histogram become increasingly separated and the width of the signal distribution increases as the square root of N_{b+d}, consistent with kinetic Monte Carlo simulations.

Further, in some embodiments assay conditions are controlled to tune the kinetic behavior to improve discrimination of query probe binding events to the target analyte from background binding. For example, in some embodiments the technology comprises control of assay conditions such as, e.g., using a query probe that is designed to interact weakly with the target analyte (e.g., in the nanomolar affinity range); controlling the temperature such that the query probe interacts weakly with the target analyte; controlling the solution conditions, e.g., ionic strength, ionic composition, addition of chaotropic agents, addition of competing probes, and/or addition of molecular crowding agents.

### Analytes

The technology is not limited in the analyte that is detected, quantified, identified, or otherwise characterized (e.g., presence, absence, amount, concentration, state). The term “analyte” as used herein is a broad term and is used in its ordinary sense, including, without limitation, to refer to a substance or chemical constituent in a sample such as a biological fluid (for example, blood, interstitial fluid, cerebral spinal fluid, lymph fluid or urine) that can be analyzed. In some embodiments, samples comprise multiple substances or chemical constituents that are of the same and/or different types. Accordingly, in some embodiments, the terms “target analyte” and “non-target analyte” are used to differentiate an analyte that is the object of detection, quantification, identification, or characterization (e.g., presence, absence, amount, concentration, state) by an assay from other substance or chemical constituents that may be the same type as the analyte but which are not the object of detection, quantification, identification, or characterization (e.g., presence, absence, amount, concentration, state) by the assay. Thus, when used herein, the term “target analyte” refers to an analyte that is the object of detection, quantification, identification, or characterization (e.g., presence, absence, amount, concentration, state) by an assay. The term “non-target analyte” refers to a substance or chemical constituent of a sample that is not the object of detection, quantification, identification, or characterization (e.g., presence, absence, amount, concentration, state) by an assay. The “non-target analyte” may or may not be the same type of substance or chemical constituent as the analyte.

Analytes can include naturally occurring substances, artificial substances, metabolites, and/or reaction products. In some embodiments, the analyte comprises a salt, sugars, protein, fat, vitamin, or hormone. In some embodiments, the analyte is naturally present in a biological sample (e.g., is “endogenous”); for example, in some embodiments, the analyte is a metabolic product, a hormone, an antigen, an antibody, and the like. Alternatively, in some embodiments, the analyte is introduced into a biological organism (e.g., is “exogenous”), for example, a drug, drug metabolite, a drug precursor (e.g., prodrug), a contrast agent for imaging, a radioisotope, a chemical agent, etc. The metabolic products of drugs and pharmaceutical compositions are also contemplated analytes.

In some embodiments, the analyte is a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a combination of one or more of a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc.

In some embodiments, the analyte is part of a multimolecular complex, e.g., a multiprotein complex, a nucleic acid/protein complex, a molecular machine, an organelle (e.g., a cell-free mitochondrion, e.g., in plasma; a plastid; golgi, endoplasmic reticulum, vacuole, peroxisome, lysosome, and/or nucleus), cell, virus particle, tissue, organism, or any macromolecular complex or structure or other entity that can be captured and/or detected and that is amenable to analysis by the technology described herein (e.g., a ribosome, spliceosome, vault, proteasome, DNA polymerase III holoenzyme, RNA polymerase II holoenzyme, symmetric viral capsids, GroEL/GroES; membrane protein complexes: photosystem I, ATP synthase, nucleosome, centriole and microtubule-organizing center (MTOC), cytoskeleton, flagellum, nucleolus, stress granule, germ cell granule, or neuronal transport granule). For example, in some embodiments, a multimolecular complex is isolated and the technology finds use in characterizing, identifying, quantifying, and/or detecting one or more molecules (analytes) associated with (e.g., that is a component of) the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

In some embodiments, the analyte is chemically modified to provide a site for query probe binding. For instance, in some embodiments, beta-elimination of phosphoserine and phosphothreonine under strongly basic conditions is used to introduce an alkene, followed by Michael addition of a nucleophile such as a dithiol to the alkene. The remaining free thiol is then used for conjugation to a maleimide-containing oligonucleotide with a sequence complementary to an oligonucleotide query probe. The post-translational modifications phosphoserine and phosphothreonine may then be probed using the query probe and analyzed as described herein.

As used herein “detect an analyte” or “detect a substance” will be understood to encompass direct detection of the analyte itself or indirect detection of the analyte by detecting its by-product(s).

### Capture

Embodiments of the technology comprise capture of an analyte. In some embodiments, the analyte is captured and immobilized. In some embodiments, the analyte is stably attached to a solid support. In some embodiments, the solid support is immobile relative to a bulk liquid phase contacting the solid support. In some embodiments, the solid support is diffusible within a bulk liquid phase contacting the solid support.

In some embodiments, stable attachment of the target analyte to a surface or other solid substrate is provided by a high-affinity or irreversible interaction (e.g., as used herein, an “irreversible interaction” refers to an interaction having a dissociation half-life longer than the observation time, e.g., in some embodiments, a time that is 1 to 5 minutes (e.g., 60, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer). The technology is not limited in the components and/or methods used for capture of the analyte. For example, the stable attachment is provided by a variety of methods, including but not limited to one or more of the following.

In some embodiments, an analyte is immobilized by a surface-bound capture probe with a dissociation constant (K_{D}) for the analyte smaller than approximately 1 nanomolar (nM) (e.g., less than 1.5, 1.4, 1.3, 1.2, 1.1, 1.0, 0.9, 0.8, 0.7, 0.6, 0.5 nanomolar) and a dissociation rate constant for the analyte that is smaller than approximately 1 min⁻¹ (e.g., less than approximately 1.5, 1.4, 1.3, 1.2, 1.1, 1.0, 0.9, 0.8, 0.7, 0.6, 0.5 min⁻¹). Exemplary surface-bound capture probes include, e.g., an antibody, antibody fragment, nanobody, or other protein; a high-affinity DNA-binding protein or ribonucleoprotein complex such as Cas9, dCas9, Cpf1, transcription factors, or transcription activator-like effector nucleases (TALENs); a nucleic acid such as a double-stranded oligonucleotide, a single-stranded oligonucleotide, an aptamer; a small organic molecule; or a metal ion complex.

In some embodiments, an analyte is immobilized by direct noncovalent attachment to a surface (e.g., by interactions between the analyte and the surface, e.g., a glass surface or a nylon, nitrocellulose, or polyvinylidene difluoride membrane).

In some embodiments, an analyte is immobilized by chemical linking (e.g., by a covalent bond) of the analyte to the solid support. In some embodiments, the analyte is chemically linked to the solid support by, e.g., a carbodiimide, a N-hydroxysuccinimide esters (NHS) ester, a maleimide, a haloacetyl group, a hydrazide, or an alkoxyamine. In some embodiments, an analyte is immobilized by radiation (e.g., ultraviolet light)-induced cross-linking of the target analyte to the surface and/or to a capture probe attached to the surface. In some embodiments, the capture probe is a rabbit monoclonal antibody. In some embodiments in which the analyte comprises a carbohydrate or polysaccharide, the capture probe comprises a carbohydrate-binding protein such as a lectin or a carbohydrate-binding antibody.

Alternatively, instead of immobilizing the target analyte to a solid support that is relatively stationary with respect to a bulk phase that contacts the solid support as described above, some embodiments provide that the target analyte is associated with a freely diffusing particle that diffuses within the bulk fluid phase contacting the freely diffusing particle. Accordingly, in some embodiments, the target analyte is covalently or noncovalently bound to a freely diffusing substrate. In some embodiments, the freely diffusing substrate is, e.g., a colloidal particle (e.g., a particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)). In some embodiments, the freely diffusing substrate comprises and/or is made of, e.g., polystyrene, silica, dextran, gold, or DNA origami. In some embodiments, the target analyte is associated with a freely diffusing particle that diffuses slowly relative to the diffusion of the query probe, e.g., the target analyte has a diffusion coefficient that is less than approximately 10% (e.g., less than 15, 14, 13, 12, 11, 10.5, 10.4, 10.3, 10.2, 10.1, 10.0, 9.9, 9.8, 9.7, 9.6, 9.5, or 9.0% or less) of the diffusion coefficient of the query probe.

Furthermore, in some embodiments the target analyte is associated with a freely diffusing particle and the location of the target analyte is observable and/or recordable independently of observing and/or recording query probe binding. For example, in some embodiments a detectable label (e.g., a fluorophore, fluorescent protein, quantum dot) is covalently or noncovalently attached to the target analyte, e.g., for detection and localization of the target analyte. Accordingly, in some embodiments the position of the target analyte and the position of query probe binding events are simultaneously and independently measured.

### Query

Embodiments of the technology comprise a query probe (e.g., a detectably labeled query probe) that binds transiently and repeatedly to the analyte, e.g., a query probe that binds to and dissociates from the target analyte several (e.g., greater than 1, 2, 3, 4, 5, 6, 7, 8, 9, 10, or more) times per observation window. In some embodiments, the query probe has a dissociation constant (K_{D}) for the analyte of larger than approximately 1 nanomolar (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more nanomolar) under the assay conditions. In some embodiments, the query probe has a binding and/or a dissociation constant for the analyte that is larger than approximately 1 min⁻¹ (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more min⁻¹). In some embodiments, the query probe is a fluorogenic query probe as described herein.

The technology is not limited in the query probe. In some embodiments, the query probe is an antibody or antibody fragment. In some embodiments, the query probe is a low-affinity antibody or antibody fragment. In some embodiments, the query probe is a nanobody, a DNA-binding protein or protein domain, a methylation binding domain (MBD), a kinase, a phosphatase, an acetylase, a deacetylase, an enzyme, or a polypeptide. In some embodiments, the query probe is an oligonucleotide that interacts with the target analyte. For example, in some embodiments the query probe is an oligonucleotide that hybridizes to the target analyte to form a duplex that has a melting temperature that is within approximately 10 degrees Celsius of the temperature at which the observations are made (e.g., approximately 7-12 nucleotides for observation that is performed at room temperature). In some embodiments, the query probe is an aptamer (e.g., as described in U.S. Pat. App. Ser. No. 63/389,406, which is incorporated herein by reference). In some embodiments, the query probe is a fluorogenic probe (e.g., as described herein). In some embodiments, the query probe is a mononucleotide.

In some embodiments, the query probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments, the query probe is a pharmaceutical agent, e.g., a drug or other bioactive molecule. In some embodiments, the query probe is a metal ion complex. In some embodiments, the query probe is a methyl-binding domain (e.g., MBD1). In some embodiments, the query probe is labeled with a detectable label as described herein. In some embodiments, the query probe is covalently linked to the detectable label. In some embodiments, the query probe is indirectly and/or non-covalently linked and/or associated with the detectable label. In some embodiments, the detectable label is fluorescent.

In some embodiments, the query probe is a fluorogenic probe comprising a detectable label (e.g., a fluorescent moiety) and a quencher of the detectable label, e.g., as further described herein and in the Examples.

In some embodiments, the query probe is a mouse monoclonal antibody.

In some embodiments in which the analyte comprises a carbohydrate or polysaccharide, the query probe comprises a carbohydrate-binding protein such as a lectin or a carbohydrate-binding antibody.

In some embodiments, the query probe is a dye that binds to (e.g., associates with) an analyte. For example, in some embodiments, the query probe is a fluorogenic dye (e.g., a fluorescent dye that produces a fluorescent signal when associated with an analyte that is brighter than the fluorescent signal produced by the dye when dissociated from the analyte) that associates with an analyte and dissociates from the analyte with acceptable kinetics for use in SiMREPS technologies.

### Detection

The technology provides for the detection of target analytes, e.g., in the presence of similar analytes and, in some embodiments, background noise. In some embodiments, signal originating from the transient binding of the query probe to the target analyte is distinguishable from the signal produced by unbound query probe (e.g., by observing, monitoring, and/or recording a localized change in signal intensity during the binding event). In some embodiments, observing the transient binding of the query probe (e.g., a fluorescently labeled query probe) to the target analyte is provided by a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the technology provided herein uses query probes having a fluorescence emission that is quenched when not bound to the target analyte and/or a fluorescence emission that is dequenched when bound to the target analyte, e.g., a fluorogenic probe as described herein.

The technology comprises locating and/or observing the transient binding of a query probe to an analyte within a discrete region of an area and/or a discrete region of a volume that is observed, e.g., at particular spatial coordinates in a plane or a volume. In some embodiments, the error in determining the spatial coordinates of a binding or dissociation event (e.g., due to limited signal, detector noise, or spatial binning in the detector) is small (e.g., minimized, eliminated) relative to the average spacing between immobilized (e.g., surface-bound) target analytes. In some embodiments comprising use of wide-field fluorescence microscopy, measurement errors are minimized and/or eliminated by use of effective detector pixel dimensions in the specimen plane that are not larger than the average distance between immobilized (e.g., surface-bound) target analytes and that many fluorescent photons (in some embodiments, more than 100, e.g., more than 80, 85, 90, 95, 100, 105, 110, 115, 120, 125, or 130 or more) are collected per time point of detection.

In some embodiments, the detectable (e.g., fluorescent) query probe produces a fluorescence emission signal when it is close to the surface of the solid support (e.g., within about 100 nm of the surface of the solid support). When unbound, query probes quickly diffuse and thus are not individually detected; accordingly, when in the unbound state, the query probes produce a low level of diffuse background fluorescence. Consequently, in some embodiments detection of bound query probes comprises use of total internal reflection fluorescence microscopy (TIRF), HiLo microscopy (see, e.g., US20090084980, EP2300983 B1, WO2014018584 A1, WO2014018584 A1, each of which is incorporated herein by reference), confocal scanning microscopy, or other technologies comprising illumination schemes that illuminate (e.g., excite) only those query probe molecules near or on the surface of the solid support. Thus, in some embodiments, only query probes that are bound to an immobilized target near or on the surface produce a point-like emission signal (e.g., a “spot”) that can be confirmed as originating from a single molecule.

In some embodiments, the query probe comprises a fluorescent label having an emission wavelength. Detection of fluorescence emission at the emission wavelength of the fluorescent label indicates that the query probe is bound to an immobilized target analyte. Binding of the query probe to the target analyte is a “binding event”. In some embodiments of the technology, a binding event has a fluorescence emission having a measured intensity greater than a defined threshold. For example, in some embodiments a binding event has a fluorescence intensity that is above the background fluorescence intensity (e.g., the fluorescence intensity observed in the absence of a target analyte). In some embodiments, a binding event has a fluorescence intensity that is at least 1, 2, 3, 4 or more standard deviations above the background fluorescence intensity (e.g., the fluorescence intensity observed in the absence of a target analyte). In some embodiments, a binding event has a fluorescence intensity that is at least 2 standard deviations above the background fluorescence intensity (e.g., the fluorescence intensity observed in the absence of a target analyte). In some embodiments, a binding event has a fluorescence intensity that is at least 1.5, 2, 3, 4, or 5 times the background fluorescence intensity (e.g., the mean fluorescence intensity observed in the absence of a target analyte).

Accordingly, in some embodiments detecting fluorescence at the emission wavelength of the query probe that has an intensity above the defined threshold (e.g., at least 2 standard deviations greater than background intensity) indicates that a binding event has occurred (e.g., at a discrete location on the solid support where a target analyte is immobilized). Also, in some embodiments detecting fluorescence at the emission wavelength of the query probe that has an intensity above the defined threshold (e.g., at least 2 standard deviations greater than background intensity) indicates that a binding event has started. Accordingly, in some embodiments detecting an absence of fluorescence at the emission wavelength of the query probe that has an intensity above the defined threshold (e.g., at least 2 standard deviations greater than background intensity) indicates that a binding event has ended (e.g., the query probe has dissociated from the target analyte). The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the target analyte (e.g., an on/off event), e.g., a query probe dissociating from a bound state or a query probe associating with a target analyte from the unbound state.

Methods according to the technology comprise counting the number of query probe binding events that occur at each discrete location (e.g., at a position identified by x, y coordinates) on the solid support during a defined time interval that is the “acquisition time” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 0 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes).

Further, the length of time the query probe remains bound to the target analyte during a binding event is the “dwell time” of the binding event. The number of binding events detected during the acquisition time and/or the lengths of the dwell times recorded for the binding events is/are characteristic of a query probe binding to a target analyte and thus provide an indication that the target analyte is immobilized at said discrete location and thus that the target analyte is present in the sample.

Binding of the query probe to the immobilized target analyte and/or and dissociation of the query probe from the immobilized target analyte is/are monitored (e.g., using a light source to excite the fluorescent probe and detecting fluorescence emission from a bound query probe, e.g., using a fluorescence microscope) and/or recorded during a defined time interval (e.g., during the acquisition time). The number of times the query probe binds to the nucleic acid during the acquisition time and/or the length of time the query probe remains bound to the nucleic acid during each binding event and the length of time the query probe remains unbound to the nucleic acid between each binding event (e.g., the “dwell times” in the bound and unbound states, respectively) are determined, e.g., by the use of a computer and software (e.g., to analyze the data using a hidden Markov model and Poisson statistics).

In some embodiments, positive and/or negative control samples are measured (e.g., a control sample known to comprise or not to comprise a target). Fluorescence detected in a negative control sample is “background fluorescence” or “background (fluorescence) intensity” or “baseline”.

In some embodiments, data comprising measurements of fluorescence intensity at the emission wavelength of the query probe are recorded as a function of time. In some embodiments, the number of binding events and the dwell times of binding events (e.g. for each immobilized analyte) are determined from the data (e.g., by determining the number of times and the lengths of time the fluorescence intensity is above a threshold background fluorescence intensity). In some embodiments, transitions (e.g., binding and dissociation of a query probe) are counted for each discrete location on the solid support where a target analyte is immobilized. In some embodiments, a threshold number of transitions is used to discriminate the presence of a target analyte at a discrete location on the solid support from background signal, non-target analyte, and/or spurious binding of the query probe.

In some embodiments, a distribution of the number of transitions for each immobilized target is determined—e.g., the number of transitions is counted for each immobilized analyte observed. In some embodiments a histogram is produced. In some embodiments, characteristic parameters of the distribution are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer.

Pattern recognition (e.g., using training sets, supervised learning, unsupervised learning, and analysis of unknown samples) associates identified patterns with analytes such that particular patterns (e.g., fluorescence intensity detected at one or more emission wavelengths as a function of time) provide a “kinetic fingerprint” of particular analytes that find use in detection, quantification, and identification of analytes.

In some embodiments, the distribution produced from a target analyte is significantly different than a distribution produced from a non-target analyte or the distribution produced in the absence of a target analyte. In some embodiments, a mean number of transitions is determined for the plurality of immobilized target analytes. In some embodiments, the mean number of transitions observed for a sample comprising a target analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time at each discrete location on the solid support. In some particular embodiments, a relatively constant probability of a transition event occurring as a function of time at a discrete location on the solid support indicates the presence of a target analyte at said discrete location on the solid support. In some embodiments, a correlation coefficient relating event number and elapsed time is calculated from the probability of a transition event occurring as a function of time at a discrete location on the solid support. In some embodiments, a correlation coefficient relating event number and elapsed time greater than 0.95 when calculated from the probability of a transition event occurring as a function of time at a discrete location on the solid support indicates the presence of a target analyte at said discrete location on the solid support.

In some embodiments, dwell times of bound query probe (τₒₙ) and unbound query probe (τ_{off}) are used to identify the presence of an analyte in a sample and/or to distinguish a sample comprising a target analyte from a sample comprising a non-target analyte and/or not comprising the analyte. For example, the τₒₙ for a target analyte is greater than the τₒₙ for a non-target analyte; and, the τ_{off} for a target analyte is smaller than the τ_{off} for a non-target analyte. In some embodiments, measuring τₒₙ and τ_{off} for a negative control and for a sample indicates the presence or absence of the analyte in the sample. In some embodiments, a plurality of τₒₙ and τ_{off} values is determined for each of a plurality of spots imaged on a solid support, e.g., for a control (e.g., positive and/or negative control) and a sample suspected of comprising an analyte. In some embodiments, a mean τₒₙ and/or τ_{off} is determined for each of a plurality of spots imaged on a solid support, e.g., for a control (e.g., positive and/or negative control) and a sample suspected of comprising a analyte. In some embodiments, a plot of τₒₙ versus τ_{off} (e.g., mean τₒₙ and τ_{off}, time-averaged τₒₙ and τ_{off}, etc.) for all imaged spots indicates the presence or absence of the analyte in the sample.

### Fluorescent Moieties

In some embodiments, a query probe comprises a fluorescent moiety (e.g., also known as a “fluorophore” or a “fluor”). A wide variety of fluorescent moieties is known in the art and methods are known for linking a fluorescent moiety to query probes.

Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4, 7, 2′, 7′-tetrachlorofluorescein (TET), 5- or 6-carboxy-4′5′2′4′5′7′ hexachlorofluorescein (HEX), 5′ or 6′-carboxy-4′,5′-dichloro-2,′7′-dimethoxyfluorescein (JOE), 5-carboxy-2′,4′,5′,7′-tetrachlorofluorescein (ZOE), rhodol, rhodamine, tetramethylrhodamine (TAMRA), 4,7-dlchlorotetramethyl rhodamine (DTAMRA), rhodamine X (ROX), and Texas Red. Examples of cyanine dyes that may find use with the present invention include but are not limited to Cy 3, Cy 3B, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

In some embodiments, the fluorescent moiety comprises a fluorescent protein (e.g., a green fluorescent protein (GFP), a modified derivative of GFP (e.g., a GFP comprising S65T, an enhanced GFP (e.g., comprising F64L)), or others known in the art such as, e.g., blue fluorescent protein (e.g., EBFP, EBFP2, Azurite, mKalama1), cyan fluorescent protein (e.g., ECFP, Cerulean, CyPet, mTurquoise2), and yellow fluorescent protein derivatives (e.g., YFP, Citrine, Venus, YPet). Embodiments provide that the fluorescent protein may be covalently or noncovalently bonded to one or more query probes, analytes, and/or capture probes.

Fluorescent dyes include, without limitation, d-Rhodamine acceptor dyes including Cy 5, dichloro[R110], dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyflporphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof.

Several classes of fluorogenic dyes and specific compounds are known that are appropriate for particular embodiments of the technology: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin. In some embodiments the fluorescent moiety a dye that is xanthene, fluorescein, rhodamine, BODIPY, cyanine, coumarin, pyrene, phthalocyanine, phycobiliprotein, ALEXA FLUOR® 350, ALEXA FLUOR® 405, ALEXA FLUOR® 430, ALEXA FLUOR® 488, ALEXA FLUOR® 514, ALEXA FLUOR® 532, ALEXA FLUOR® 546, ALEXA FLUOR® 555, ALEXA FLUOR® 568, ALEXA FLUOR® 568, ALEXA FLUOR® 594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, or a squaraine dye. In some embodiments, the label is a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference in its entirety.

In some embodiments the label (e.g., a fluorescently detectable label) is one available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, all of which are incorporated herein by reference (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO740).

One of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

### Quencher Moieties

In some embodiments, a query probe (e.g., a fluorogenic probe) comprises a quencher moiety. A wide variety of quencher moieties is known in the art and methods are known for linking a quencher moiety to a query probe. As used herein, “quenching group” or “quencher moiety” and similar terms refers to any fluorescence-modifying group that can attenuate, at least partly, the energy (e.g., light) emitted by a fluorescent moiety. This attenuation is referred to herein as “quenching”. Hence, irradiation of the fluorescent moiety in the presence of the quencher moiety leads to an emission signal from the fluorescent moiety that is less intense than expected, or even completely absent. Quenching typically occurs through energy transfer between the fluorescent moiety and the quencher moiety or by ground state stabilization wherein the quencher and fluorophore form a complex that prevents or inhibits excitation of the fluorescent moiety and thus minimizes and/or eliminates fluorescence by the fluorescent moiety.

Further, the technology is not limited in the type, structure, or composition of the quencher moiety. Exemplary quenching moieties include a Black Hole Quencher, an Iowa Black Quencher, and derivatives, modifications thereof, and related moieties. Exemplary quenching moieties include BHQ-0, BHQ-1, BHQ-2, and BHQ-3. Further examples of quenchers include colloidal nanocrystals (e.g., quantum dots and gold nanoparticles) that can quench fluorescence through energy transfer. In addition, in some embodiments, nonspecific interactions between fluorophores and nearby molecules (e.g., proteins, DNA duplexes) produce quenching (see, e.g., Hwang (2014) Chem Soc Rev 43: 1221-29, incorporated herein by reference). In some embodiments, any molecule, moiety, or atom in the local environment of a fluorescent moiety that produces fluorescence quenching or enhancement (e.g., by modifying the molecular and/or electronic structure of the fluorescent moiety) finds use in the technology described herein.

In some embodiments, fluorescent moiety-quencher moiety include, e.g., DLO-FB1 (5′-FAM/3′-BHQ-1) DLO-TEB1 (5′-TET/3′-BHQ-1), DLO-JB1 (5′-JOE/3′-BHQ-1), DLO-1-HB1 (5′-HEX/3′-BHQ-1), DLO-C3B2 (5′-Cy3/3′-BHQ-2), DLO-TAB2 (5′-TAMRA/3′-BHQ-2), DLO-RB2 (5′-ROX/3′-BHQ-2), DLO-C5B3 (5′-Cy5/3′-BHQ-3), DLO-C55B3 (5′-Cy5.5/3′-BHQ-3), MBO-FB1(5′-FAM/3′-BHQ-1), MBO-TEB1 (5′-TET/3′-BHQ-1), MBO-JB1 (5-JOE/3′-BHQ-1), MBO-HB1 (5′-HEX/3′-BHQ-1), MBO-C3B2 (5′-Cy3/3′-BHQ-2), MBO-TAB2 (5′-TAMRA/3′-BHQ-2), MBO-RB2 (5′-ROX/3′-BHQ-2); MBO-C5B3 (5′-Cy5/3′-BHQ-3), MBO-C55B3 (5′-Cy5.5/3′-BHQ-3) or similar FRET pairs available from Biosearch Technologies, Inc. of Novato, Calif. See, e.g., U.S. Pat. App. Pub. No. US20100317005 incorporated herein by reference.

### Methods

Some embodiments provide a method of identifying an analyte by repetitive query probe binding. In some embodiments, methods comprise immobilizing an analyte to a solid support. In some embodiments, the solid support is a surface (e.g., a substantially planar surface, a rounded surface), e.g., a surface in contact with a bulk solution, e.g., a bulk solution comprising analyte. In some embodiments, the solid support is a freely diffusible solid support (e.g., a bead, a colloidal particle, e.g., a colloidal particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)), e.g., that freely diffuses within the bulk solution, e.g., a bulk solution comprising the analyte. In some embodiments, immobilizing an analyte to a solid support comprises covalent interaction between the solid support and analyte. In some embodiments, immobilizing an analyte to a solid support comprises non-covalent interaction between the solid support and analyte. In some embodiments, immobilizing an analyte to a solid support comprising stably binding the analyte to a surface-bound capture probe.

In some embodiments, the analyte (e.g., a molecule, e.g., a molecule such as, e.g., a protein, peptide, nucleic acid, small molecule, lipid, metabolite, drug, etc.) is stably immobilized to a surface and methods comprise repetitive (e.g., transient, low-affinity) binding of a query probe to the target analyte. In some embodiments, methods comprise detecting the repetitive (e.g., transient, low-affinity) binding of a query probe to the target analyte. In some embodiments, methods comprise generating a dataset comprising a signal produced from query probe binding to the analyte (e.g., a dataset of query probe signal as a function of time) and information (e.g., coordinates, e.g., x, y coordinates) describing the spatial position on the surface of the query probe binding to the analyte. In some embodiments, the dataset is processed (e.g., manipulated, transformed, visualized, etc.), e.g., to improve the spatial resolution of the query probe binding events. For example, in particular embodiments, the dataset (e.g., comprising query probe signal as a function of time and information (e.g., coordinates, e.g., x, y coordinates) describing the spatial position on the surface of the query probe binding to the analyte) is subjected to processing. In some embodiments, the processing comprises a frame-by-frame subtraction process to generate differential intensity profiles showing query probe binding or dissociation events within each frame of the time series data. Data collected during the development of the technology described herein indicate that the differential intensity profiles have a higher resolution than the query probe binding signal vs. position map. In some embodiments, after determining the spatial position (e.g., x, y coordinates) of each query probe binding and/or dissociation event, a plurality of events is clustered according to spatial position and the kinetics of the events within each cluster are subjected to statistical analysis to determine whether the cluster of events originates from a given target analyte.

For instance, some embodiments of methods for quantifying one or more surface-immobilized or diffusing target analytes comprise one or more steps including, e.g., measuring the signal of one or more transiently binding query probes to the immobilized target analyte(s) with single-molecule sensitivity. In some embodiments, methods comprise tracking (e.g., detecting and/or recording the position of) target analytes independently from query probe binding. In some embodiments, the methods further comprise calculating the time-dependent probe binding signal intensity changes at the surface as a function of position (e.g., x, y position). In some embodiments, calculating the time-dependent query probe binding signal intensity changes at the surface as a function of position (e.g., x, y position) produces a “differential intensity profile” for query probe binding to the analyte. In some embodiments, the methods comprise determining the position (e.g., x, y position) of each query probe binding and dissociation event (“event”) with sub-pixel accuracy from a differential intensity profile. In some embodiments, methods comprise grouping events into local clusters by position (e.g., x, y position) on the surface, e.g., to associate events for a single immobilized target analyte. In some embodiments, the methods comprise calculating kinetic parameters from each local cluster of events to determine whether the cluster originates from a particular analyte, e.g., from transient probe binding to a particular analyte.

Embodiments of methods are not limited in the analyte that is detected. For example, in some embodiments the analyte is polypeptide, e.g., a protein or a peptide. In some embodiments, the target analyte is a nucleic acid. In some embodiments, the target analyte is a small molecule.

In some embodiments, the interaction between the target analyte and the query probe is distinguishably influenced by a covalent modification of the target analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, the analyte is a nucleic acid comprising a covalent modification to a nucleobase, a ribose, or a deoxyribose moiety of the target analyte.

In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid. In some embodiments, the query probe is a nucleic acid aptamer. In some embodiments, the aptamer is optimized (e.g., in length and/or base composition) as described in U.S. Pat. App. Ser. No. 63/389,406, which is incorporated herein by reference.

In some embodiments, the query probe is a low-affinity antibody, antibody fragment, or nanobody.

In some embodiments, the query probe is a DNA-binding protein, RNA-binding protein, or a DNA-binding ribonucleoprotein complex.

In some embodiments, the position, e.g., the (x,y) position, of each binding or dissociation event is determined by subjecting the differential intensity profile to centroid determination, least-squares fitting to a Gaussian function, least-square fitting to an airy disk function, least-squares fitting to a polynomial function (e.g., a parabola), or maximum likelihood estimation.

In some embodiments, the capture probe is a high-affinity antibody, antibody fragment, or nanobody. In some embodiments, the capture probe is a nucleic acid. In some embodiments, the capture probe is an aptamer. In some embodiments, capture is mediated by a covalent bond cross-linking the target analyte to the surface. In some embodiments, the target analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

### Design of Fluorogenic Probes

In some embodiments, fluorogenic probes are designed for fluorogenic SiMREPS. In some embodiments, the technology described herein provides a method for designing fluorogenic SiMREPS probes. In some embodiments, methods comprise providing a query probe nucleotide sequence of approximately 15 nt (e.g., 13, 14, 15, 16, or 17 nt) that is complementary to a target nucleic acid analyte. Next, methods comprise producing a mutant sequence (“candidate probe”) from the query probe nucleotide sequence by selecting (e.g., randomly selecting) approximately 3 to 7 (e.g., 3, 4, 5, 6, or 7) nucleotides and changing the selected nucleotides to a different nucleotide base. That is, methods comprise introducing mismatched bases into the query probe nucleotide sequence to produce a candidate probe sequence. Then, methods comprise calculating the free energy of association for the candidate probe with the target analyte sequence (ΔG_{MUT}). In some embodiments, methods comprise calculating the free energy of association for the candidate probe with a target analyte sequence that is a mutant sequence (ΔG_{MUT}) and calculating the free energy of association for the candidate probe with the wild-type sequence (ΔG_{WT}) related to the mutant sequence, e.g., to provide a measure of the ability of the candidate probe to differentiate between mutant and wild-type variants of a genetic sequence (“mismatch discrimination”). In some embodiments, methods comprise calculating the free energy of secondary structures formed by the candidate probe. Candidate probes that do not form secondary structures are preferred (“secondary structure-free”).

In some embodiments, candidate probes are assigned a score for high mismatch discrimination, little or no predicted secondary structure, and high similarity between (ΔG_{MUT}) and the free energy of hybridization between the target analyte and a conventional SiMREPS probe (ΔG_{FP1}). In some embodiments, a score comparing the free energy of hybridization between the target analyte and a conventional SiMREPS probe is calculated according to Equation 1 (see Examples). In some embodiments, a score is assigned to bias the method to identify probe candidates that bind less stably to WT than MUT by setting a probe candidate score to zero if ΔG_{WT}<ΔG_{MUT}.

In some embodiments, the process is iterative and is repeated at least 100; 1,000; 100,000; or 1,000,000 times.

In some embodiments, methods comprise discarding duplicate probe candidate sequences (“replicates”). In some embodiments, methods comprise selecting probe candidate sequences with mismatches evenly distributed throughout the probe, e.g., by identifying sequences that formed no more than 3 continuous basepairs with any portion of the target strand. In some embodiments, methods comprise identifying probe candidates comprising minimal GC content to maximize fluorescence (minimize and/or eliminate fluorescence quenching by G and C bases). In some embodiments, methods comprise discarding candidate probes that self-dimerize. In some embodiments, methods comprise identifying a probe candidate that is predicted to form the smallest number of predicted self-dimer states and/or that is predicted to form the most weakly hybridized self-dimers. In some embodiments, methods comprise identifying a probe candidate that has minimal ability and/or tendency to form self-complementary structures by selecting for probe candidates comprising high AG content, high AC content, high TG content, or high TC content. As used herein, the terms “high AG content”, “high AC content”, “high TG content”, and “high TC content” refer a nucleotide base composition of a probe in which all bases of the probe are A or G, A or C, T or G, and T or C, respectively, or a nucleotide base composition of a probe in which all bases except 1 base or 2 bases are A or G, A or C, T or G, and T or C, respectively.

### Multiplex SiMREPS

Embodiments of the technology related to a multiplex SiMREPS assay for detecting, quantifying, identifying, or otherwise characterizing multiple analytes (e.g., to describe the presence, absence, amount, concentration, and/or state of multiple analytes) in a sample. As described herein, multiplex SiMREPS comprises used of capture probes and query probes to capture and assay multiple analytes simultaneously. Further, as described herein, multiplex SiMREPS comprises used of capture probes and query probes to capture and assay multiple types of analytes. In particular embodiments (e.g., as described in the Examples), antibodies are used as capture probes for protein analytes, nucleic acids are used as capture probes for nucleic acid analytes, Fab fragments are used as query probes for protein analytes, and nucleic acids are used for nucleic acid analytes. Nucleic acid query probes may be fluorogenic probes as described herein. Nucleic acid query probes may be aptamer probes as described in U.S. Pat. App. Ser. No. 63/389,406, which is incorporated herein by reference.

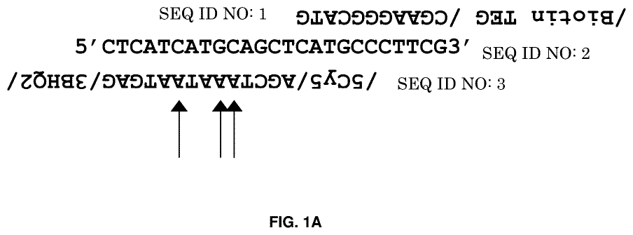

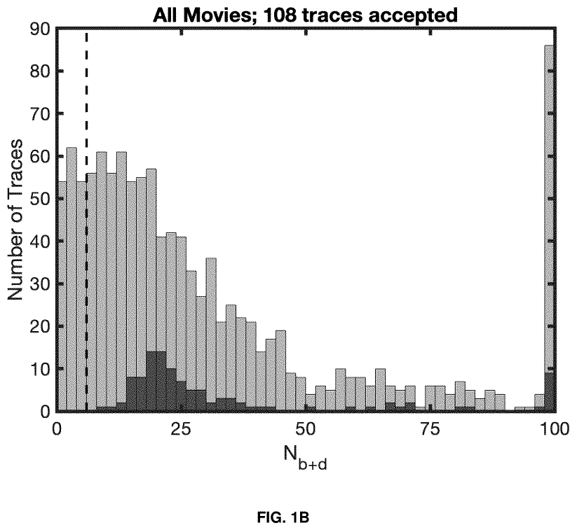

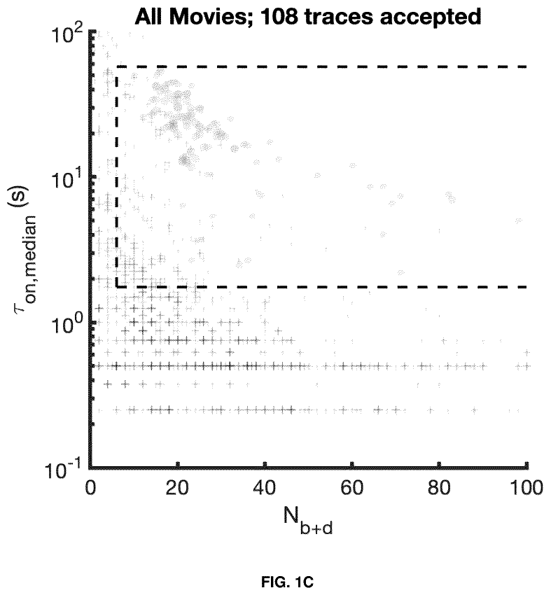

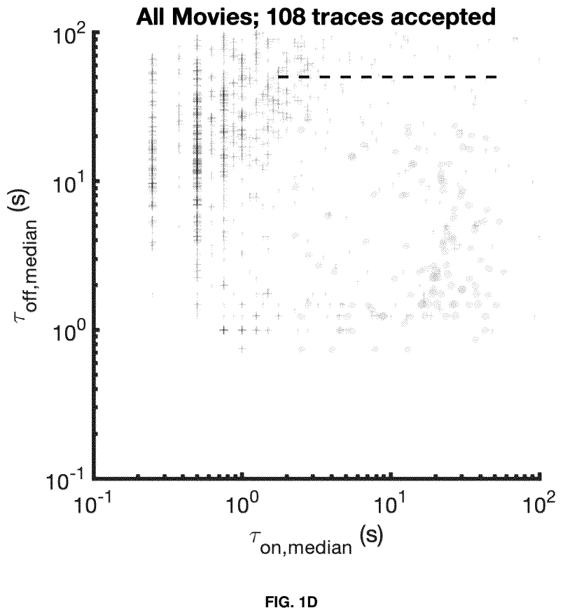

In some embodiments, multiple analytes are detected, quantified, identified, or otherwise characterized (e.g., presence, absence, amount, concentration, state) in a multiplex SiMREPS assay using analyte-specific query probes comprising different labels. In some embodiments, multiple analytes are detected, quantified, identified, or otherwise characterized (e.g., presence, absence, amount, concentration, state) in a multiplex SiMREPS assay based on differences in the time-dependent intensity signals produced from analyte-specific query probes all comprising the same label. See, e.g., Example 3 and FIG. 1n some embodiments, multiple analytes are detected, quantified, identified, or otherwise characterized (e.g., presence, absence, amount, concentration, state) in a multiplex SiMREPS assay using analyte-specific query probes comprising different labels and using differences in the time-dependent intensity signals produced from analyte-specific query probes.

The multiplex signals can be differentiated using labeling schemes providing combinations of intensity, color, mixed color, etc. For example, in some embodiments, a multiplex SiMREPS assay comprises use of a number of query probes comprising different labels and/or label combinations (e.g., different labeling schemes): e.g., a query probe comprising a plurality (e.g., two) of a first fluorescent moiety (e.g., a “doubly labeled probe”), a query probe comprising a plurality (e.g., two) of a second fluorescent moiety (e.g., a “doubly labeled probe”), a query probe comprising a first fluorescent moiety and a second fluorescent moiety (e.g., a “two-color doubly labeled probe”), a query probe comprising one of a first fluorescent moiety (e.g., a “singly labeled probe”), and/or a query probe comprising one of a second fluorescent moiety (e.g., a “singly labeled probe”). These query probes may be mixed in a multiplex assay in combinations wherein the signals produced by these probes are differentiated, e.g., a singly labeled probe (e.g., comprising a first fluorescent moiety or a second fluorescent moiety) and a doubly labeled probe (comprising a plurality of a first fluorescent moiety or a plurality of a second fluorescent moiety), a singly labeled probe comprising a first fluorescent moiety and a singly labeled probe comprising a second fluorescent moiety, and combinations thereof. In some embodiments, the probes are provided to have different kinetic behaviors (e.g., different dwell times). For example, embodiments provide that the probes have at least 2, 3, 4, 5, or more different dwell times and the signals produced by the probes can be differentiated from each other based on the differences in the dwell times. By combining differences in dwell times and different labeling schemes, higher order multiplex SiMREPS assays are provided. For instance, by combining probes having two different dwell times (D1 and D2) and two different labeling schemes (L1 and L2), four different distinct signals can be produced (D1-L1, D1-L2, D2-L1, and D2-L2) and thus up to four different analytes can be characterized by the assay. In like manner, combinations of two dwell times×three labeling schemes, three dwell times×two labeling schemes, and three dwell times×three labeling schemes are specific embodiments provided herein for, e.g., up to 6 to 9 analytes. Further embodiments provide multiplex assays comprising a number of probes designed using 1, 2, 3, 4, 5, or more dwell times×1, 2, 3, 4, 5, or more labeling schemes to provide multiplex assays to detect from 1 to 25 (e.g., 1, 2, 3, 4, 5, 6, 7, 8, 9, 10, 11, 12, 13, 14, 15, 16, 17, 18, 19, 20, 21, 22, 23, 24, or 25) different analytes.

In some embodiments, multiplex SiMREPS assays comprise use of fluorogenic probes as described herein. Experiments conducted during the development of embodiments of the technology described herein indicated that fluorogenic probes minimized and/or eliminated background signal (noise) produced by unbound probes and thus multiplex assays using fluorogenic probes may be designed using an increased number of probes (e.g., relative to non-fluorogenic probes) used in the assay without increasing (e.g., without substantially and/or effectively increasing) the background signal.

Furthermore, in some embodiments, fluorogenic probes provide for increased versatility in multiplex SiMREPS assays because nucleic acid based fluorogenic probes are constructed to be longer than non-fluorogenic nucleic acid-based probes and, in some embodiments, nucleic acid-based fluorogenic probes comprise mismatches to their targets. Accordingly, the longer probe length provides a larger number of possibilities for probe design (e.g., enlarges the usable sequence space); and b) specificity is increased because an effectively larger sequence range of the target will be targeted. For example, a 14-mer fluorogenic SiMREPS probe comprising 3-4 mismatches may be designed to have a melting temperature that is similar to a regular (non-fluorogenic) SiMREPS probe that is a 9-mer. The fluorogenic probe will target the entirety of the 14-nt complementary region it spans with some specificity provided even for the unpaired nucleotides.

In some embodiments, methods comprise using kinetic filtering parameters to accept and/or reject time-dependent signal intensity data (e.g., traces or “kinetic fingerprints”) for each analyte in the multiplex SiMREPS assay, e.g., to differentiate first time-dependent signal intensity data (e.g., a first trace or a first kinetic fingerprint) for a first analyte from second time-dependent signal intensity data (e.g., a second trace or a second kinetic fingerprint) for a second analyte. In some embodiments, the primary criteria used to distinguish between the time-dependent signal intensity data of analytes comprise one or more of minimum and maximum values of N_{b+d} (number of binding and dissociation events per single-molecule trace within the 5-minute observation period); minimum and maximum values of the median of the bound-state dwell time (Tau(Bound, Median)); and/or the minimum and maximum values of the median of the unbound-state dwell time (Tau(Unbound, Median)). In some embodiments, criteria used to distinguish between the time-dependent signal intensity data of analytes comprises the coefficient of variation (C.V.) of all the bound-state or unbound-state dwell times in each single-molecule trace (Tau(Bound) C.V. and Tau(Unbound) C.V., respectively); maximum observed dwell times in the bound state or unbound state within each single-molecule trace (Maximum Individual Tau(Bound) and Maximum Individual Tau(Unbound), respectively); the minimum allowable signal-to-noise ratio for individual binding events (S/N Threshold (Event)) or for an entire single-molecule trace (S/N Threshold (Trace), respectively); and the maximum allowable intensity of the baseline signal in a single-molecule trace (Max Baseline Intensity). Thus, in some embodiments, the kinetic filtering parameter(s) comprise(s) one or more of Intensity Threshold, Max Baseline Intensity, signal to noise (S/N) Threshold (Event), S/N Threshold (Trace), Minimum N_{b+d}, Maximum N_{b+d}, Minimum Tau(Bound, Median) (s), Maximum Tau(Bound, Median) (s), Minimum Tau(Unbound, Median) (s), Maximum Tau(Unbound, Median) (s), Maximum Individual Tau(Bound) (s), Maximum Individual Tau(Unbound) (s), Maximum Tau(Bound) C.V., and/or Maximum Tau(Unbound) C.V.

### EXAMPLES

### Example 1—Fluorogenic Probes

Nucleic acids find use as markers of the biological status of an organism. For instance, cell-free nucleic acids such as microRNAs (miRNAs) and circulating tumor DNA (ctDNA) have recently emerged as sensitive and specific biomarkers of cancer and other diseases in humans. Accordingly, in some embodiments, the technology described herein provides a SiMREPS assay for the specific and ultrasensitive detection of nucleic acids using fluorescence microscopy and a fluorogenic molecular probe. In some embodiments, this assay comprises capturing nucleic acids (e.g., unlabeled nucleic acid analytes) on a solid support (e.g., a glass, fused silica surface, or bead) using a capture probe (e.g., a locked nucleic acid) that specifically binds a first segment of the analyte; and observing (e.g., recording) repeated, transient binding of a short nucleic acid (e.g., DNA) query or “imager” probe to a second segment of the target. A detector (e.g., configured and/or structured to provide time-lapse single molecule fluorescence microscopy) reveals repeated binding and unbinding of query probes to target analytes, and the dynamics of binding is used to generate a unique kinetic “fingerprint” that is be used to detect, quantify, identify, or otherwise characterize (e.g., with respect to presence, absence, amount, concentration, state) the target analyte.

In some embodiments, the technology uses a fluorogenic probe. During the experiments described herein, a fluorogenic query probe was provided by labeling a query probe on one end with a fluorescent moiety and on the other end with a quencher moiety. For this type of fluorogenic query probe, when the fluorogenic query probe is not associated with a target analyte, the fluorophore-quencher pair forms a ground-state stabilization complex and the quencher quenches the fluorescence of the fluorescent moiety. Upon association between the fluorogenic query probe and the target analyte, the configuration of the fluorogenic probe is constrained and quenching is minimized or eliminated, thus producing a large (e.g., approximately 50-fold) increase in fluorescence intensity. The technology is not, however, limited to this type of fluorogenic query probe. In some embodiments, the technology uses other forms of quenching (e.g. FRET-based quenching and/or protein induced fluorescence enhancement) alone or in combination with ground-state stabilization. For example, for quenchers such as BHQ-2, quenching occurs through both ground-state stabilization and FRET (e.g., when the fluorophore and quencher are not complexed, the excited fluorophore may still have its excited-state quenched via energy transfer to the quencher).

In some embodiments, the highly efficient quenching of unassociated query probe molecules that are free in solution drastically increases the signal-to-noise ratio of the kinetic fingerprinting relative to the parent SiMREPS technology (see, e.g., U.S. Pat. App. Pub. No. 20190048415, incorporated herein by reference) that does not utilize a quencher. Consequently, the improved signal-to-noise of SiMREPS using a fluorogenic probe increases the dynamic range, specificity, and speed of molecular analysis.

During the development of embodiments of the technology provided herein, experiments were conducted to determine a separation distance between the fluorogenic query probe fluorophore moiety and quencher moiety that provides sufficiently high fluorogenicity (that is, a large increase in fluorescence intensity upon association of the fluorogenic probe with a target analyte relative to the fluorescence intensity of the fluorogenic probe when dissociated from the target analyte). Data collected in these experiments indicated that a fluorogenic probe of approximately 15 nucleotides (e.g., 13, 14, 16, or 17 nt) provided a sufficient distance between the fluorescent moiety and the quencher moiety to provide a high fluorogenicity. Conventional query probes used in SiMREPS are approximately 8 nucleotides in length.

Further, experiments were conducted during the development of embodiments of the technology described herein to test the association and dissociation of the fluorogenic probes with target analytes and design fluorogenic probes having kinetic rate constants that are suitable for kinetic fingerprinting by the SiMREPS method. In particular, experiments were conducted in which the sequences of the ˜15-nt fluorogenic query probes were modified to provide a fluorogenic query probe that formed approximately 10 base pairs (e.g., 8, 9, 10, 11, or 12 base pairs) with the target analyte. Thus, the hybridized double-stranded nucleic acid structure comprising the target analyte and the fluorogenic query probe comprised a number of mismatches (e.g., approximately 5 mismatches (e.g., 1 to 9 mismatches depending on query probe length and base composition)) that decrease the stability of the duplex and provide kinetic rate constants appropriate for SiMREPS. Similar design principles are described in Chung (2022) “Fluorogenic DNA-PAINT for faster, low-background super-resolution imaging” Nature Methods 19: 554-59, incorporated herein by reference. However, while Chung reported use of probes similar to the fluorogenic probes described herein for super-resolution imaging purposes, the present technology described herein uses fluorogenic query probes that are optimized for kinetic fingerprinting by SiMREPS. Accordingly, embodiments of the technology provided herein improve the previously described SiMREPS assays (e.g., as described in U.S. Pat. App. Pub. No. 20190048415, incorporated herein by reference) using optimized fluorogenic probes to provide a new technology that dramatically improves signal-to-noise.

As described herein, a fluorogenic probe is a nucleic acid of approximately 15 nucleotides (e.g., 13, 14, 15, 16, or 17 nt) and comprising a fluorescent moiety at or near a first end (e.g., at or near the 3′ or 5′ end) and comprising a quencher moiety at or near a second end (e.g., at or near the 3′ end when the fluorescent moiety is at or near the 5′ end or at or near the 5′ end when the fluorescent moiety is at or near the 3′ end). Further, the fluorogenic probe is designed to comprise mismatches with the target analyte to provide fast association and dissociation interactions with the target analyte nucleic acid.

In some embodiments, the length of the probe; and/or the number and/or locations of the mismatches in fluorogenic probes are selected and optimized using computational methods. As an example of a probe sequence optimization process, during the development of embodiments of the technology, candidate fluorogenic probe sequences for detecting a mutant allele of a target analyte nucleic acid (AGCTCATCATGCAGCTCATGCCCTTCGTAGGAC (SEQ ID NO: 4), “MUT”) were obtained using a high-throughput computational screen in MATLAB 2019b. Starting from a sequence that was fully complementary to a 15 nt target site of the target MUT analyte, the process randomly selected five nucleotides and randomly mutates the selected five nucleotides to a different base. These five nucleotides did not include the nucleotide that pairs to the site of the polymorphism in the MUT nucleic acid or the two nucleotides at either terminus of the strand. Next, the free energies of hybridization of the candidate probe sequences to the MUT nucleic acid and to the wild-type allele nucleic acid (“WT”) (ΔG_{MUT} and ΔG_{WT}, respectively) were calculated using the “nnHSmm” function from DNADesign 0.02, which is a DNA duplex thermodynamic calculator that uses nearest neighbor base stacking interactions and does not consider C-T wobble base pairing. In addition, the free energy of the candidate probes' most stable secondary structures (ΔG_{2°}.) were calculated using MATLAB's built-in function “rnafold”, which sets ΔG_{2°}=0 if no thermodynamically-stable secondary structure is identified by the modeling. These metrics were then used to score each probe candidate in a manner that assigns the highest score to probe candidates having a large mismatch discrimination, little or no predicted secondary structure, and high similarity between ΔG_{MUT} and the free energy of hybridization between the MUT target nucleic acid analyte and a related query probe (CTG CAT GA/Cy5 (SEQ ID NO: 6), “FP1”) that was previously reported for detecting MUT (see, e.g., Hayward (2018) “Ultraspecific and Amplification-Free Quantification of Mutant DNA by Single-Molecule Kinetic Fingerprinting” JACS 140: 11755-62, incorporated herein by reference) (ΔG_{FP1}, which was calculated as −11.4 kcal/mol) using Equation 2:

$$score = \frac{( {( \frac{( Δ {G}_{WT} - Δ {G}_{MUT} )}{RT} )}^{2} - 1 )}{( {( \frac{( Δ {G}_{FP 1} - Δ {G}_{MUT} )}{RT} )}^{2} - 1 ) ( {( \frac{Δ G {2}^{°}}{RT} )}^{2} - 1 )} \qquad ( 2 )$$

where R is the ideal gas constant and T is temperature (set to 25° C.). Note that quenching occurs through ground-state stabilization of the probe in a linear configuration, not through formation of a hairpin. Thus, the present technology contrasts with conventional probe designs in which a fluorescent moiety and quencher moiety are brought close to one another by formation of a hairpin or other secondary structure by the unbound probe. Thus, the fluorogenic probes described herein are secondary structure-free.

To maximize the probability that a probe binds less stably to WT than MUT, the sequence's score was set to zero if ΔG_{WT}<ΔG_{MUT}. This process was repeated 100,000 times. After discarding replicates, the screen produced 49,250 unique, scored sequences for candidate probes. Of these candidate probes, 920 had a score between 8.5 and 9. From this set of candidate probes, sequences were selected that had mismatches evenly distributed throughout the probe nucleotide sequence by searching for sequences that formed no more than 3 continuous base pairs with any portion of the target strand. Twenty-six probe candidates satisfied this condition. Of this set, those with minimal GC content were selected based on the rationale that high GC content has been shown to quench fluorescence. Six of the remaining candidate probe sequences had 7 (of 15) bases as G or C. To select an optimized probe from this final set of six probe candidates, each probe candidate was analyzed using the OligoAnalyzer tool at idtdna.com to check for self-dimerization. It was found that four probe candidates formed self-dimers with a minimum free energy of −3.9 kcal/mol, and the other two formed self-dimers with a minimum free energy of −3.1 kcal/mol. Of these remaining two probe candidates, the top probe candidate was selected that exhibited the smallest number of predicted self-dimer states. The top candidate from the screen (GCCTCATTTTGTGCT; SEQ ID NO: 5) had a score of 8.63, ΔG_{MUT}=−10.75 kcal/mol, ΔG_{WT}=−8.21 kcal/mol, and ΔG_{2°}=−0. By contrast, a probe candidate that was fully complementary to the MUT target site had a free energy of hybridization of −23.21 kcal/mol.

During the development of embodiments of the technology described herein, experiments were conducted to test use of a fluorogenic query probe (e.g., in some embodiments, optimized according to a process similar to the process described above) to detect a nucleic acid comprising a mutation in the epidermal growth factor receptor (EGFR) gene that causes a substitution of methionine for threonine at amino acid 790 (T790M) in the EGFR protein. The T790M substitution is present in approximately half of lung cancer patients with acquired resistance to drug treatment with EGFR tyrosine kinase inhibitors.

In these experiments, a biotinylated capture probe was used to capture a 25-nt target analyte encoding T790M; and a fluorogenic query probe labeled at the 5′ end with Cy5 dye and comprising a black hole quencher (BHQ) at the 3′ end was used in a fluorogenic SiMREPS detection experiment. Oligonucleotide sequences are provided in Table 1.

TABLE 1
T790M experiment oligonucleotides

| | | SEQ |
|---|---|---|
| | | ID |
| Oligonucleotide | Sequence (5′ to 3′) | NO: |
| Capture probe | CGAAGGGCATG | 1 |
| T790M Target | CTCATCATGCAGCTCATGCCCTTCG | 2 |
| analyte | | |
| Fluorogenic | AGCTAAATAATGAG | 3 |
| query probe | | |
| “F-gen V4” | | |

The fluorogenic query probe “F-gen V4” had 14 nucleotides and contained 3 nucleotides that were mismatched to the T790M target analyte (Table 1; FIG. 1A). Arrows in FIG. 1A indicate the positions of mismatch between the query probe and the target analyte.

FIG. 1B-D show data collected from fluorogenic probe SiMREPS experiments in which a sample comprising 100 fM of the T790M target analyte in imaging buffer (IB) comprising an oxygen scavenger system (OSS) (“IB/OSS”: 1 mM Trolox, 5 mM 3,4-dihydroxybenzoate, and 50 nM protocatechuate dioxygenase in 4× phosphate-buffered saline (PBS)) was analyzed with 500 nM of the F-gen V4 query probe at 25° C. Video data were collected for 10 minutes. Representative kinetic traces are shown in FIG. 1E.

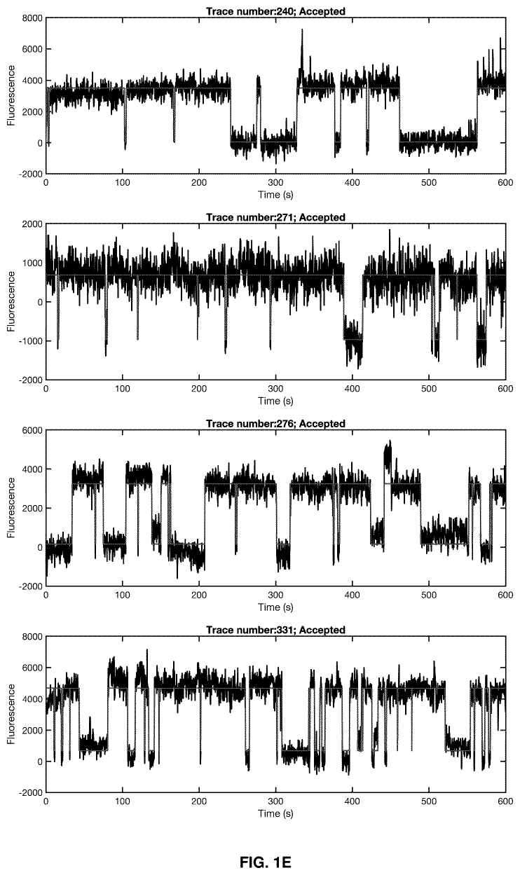

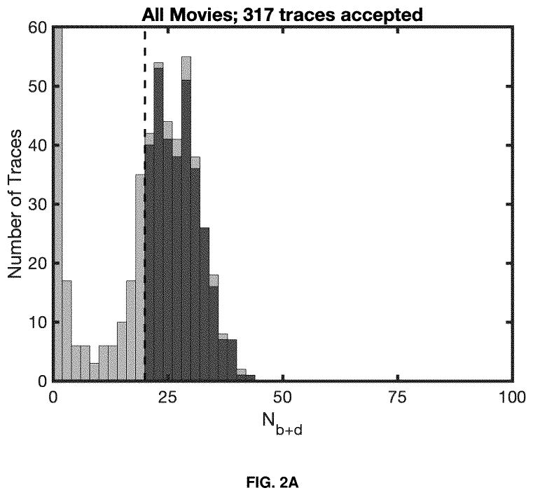

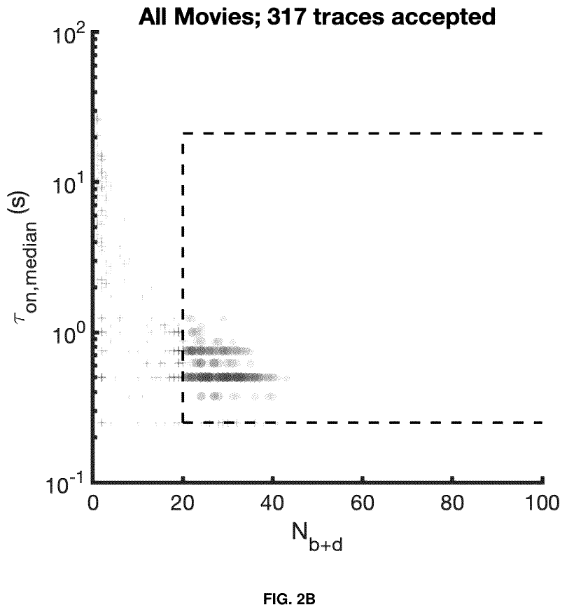

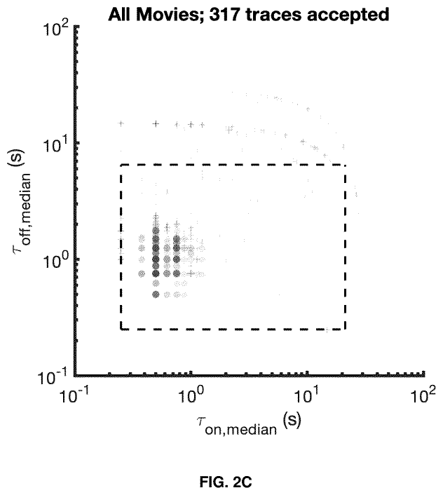

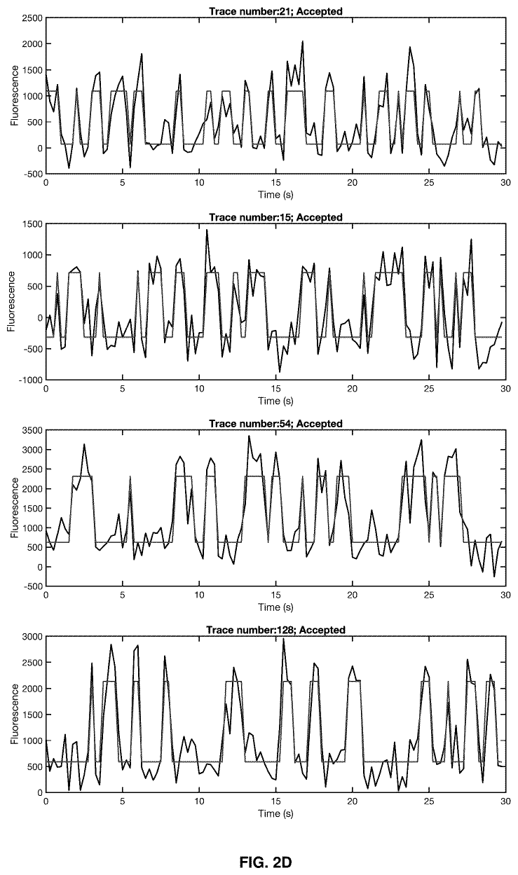

FIG. 2A-2C show data collected from fluorogenic probe SiMREPS experiments in which a sample comprising 100 fM of the T790M target analyte in IB/OSS comprising 10% w/v formamide was analyzed with 500 nM of the F-gen V4 query probe at 28° C. Video data were collected for 10 minutes. Representative kinetic traces for the first 30 seconds of the video data are shown in FIG. 2D.

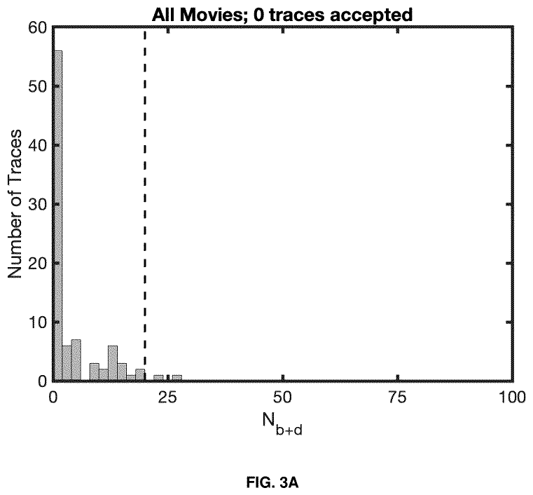

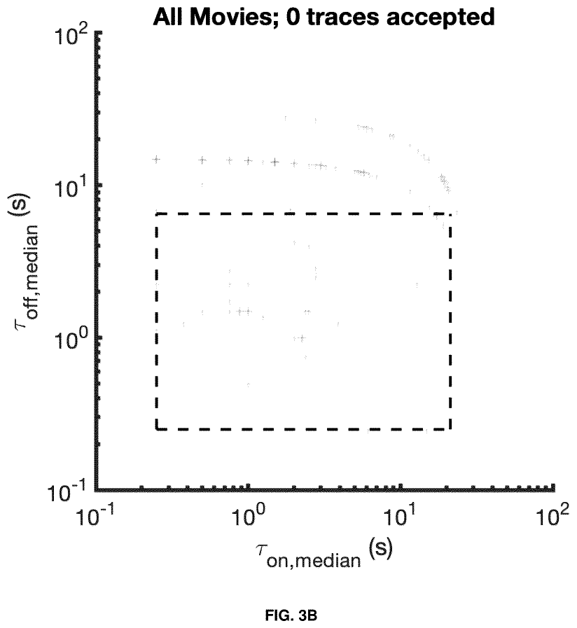

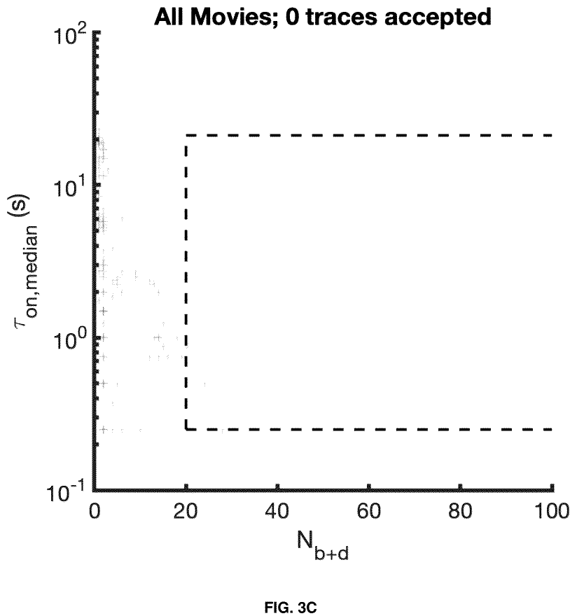

FIG. 3A-3C show data from control experiments in which a sample of IB/OSS with no target was analyzed with 500 nM of the F-gen V4 query probe. No traces were acceptable in the control experiment.

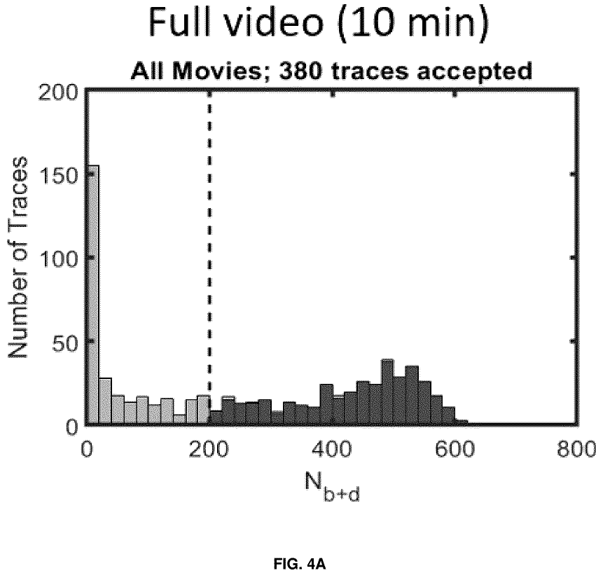

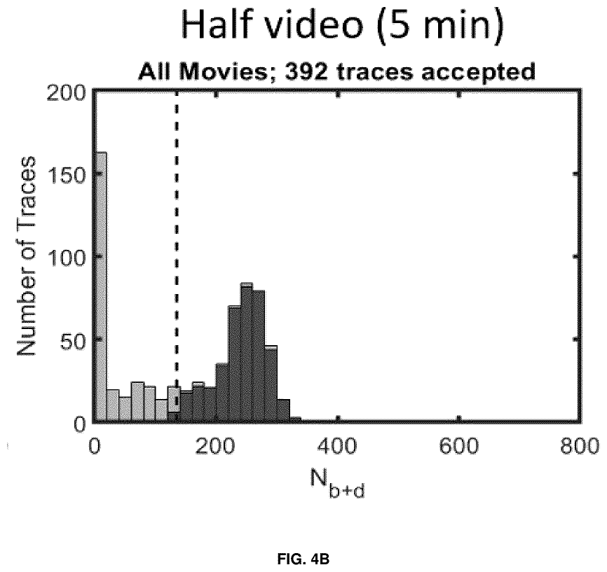

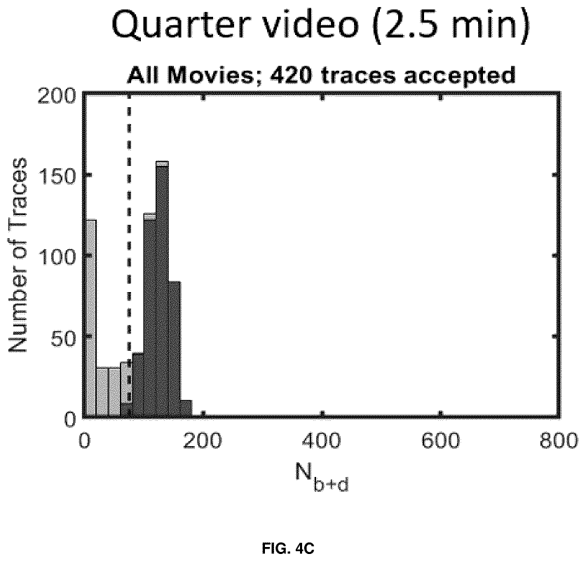

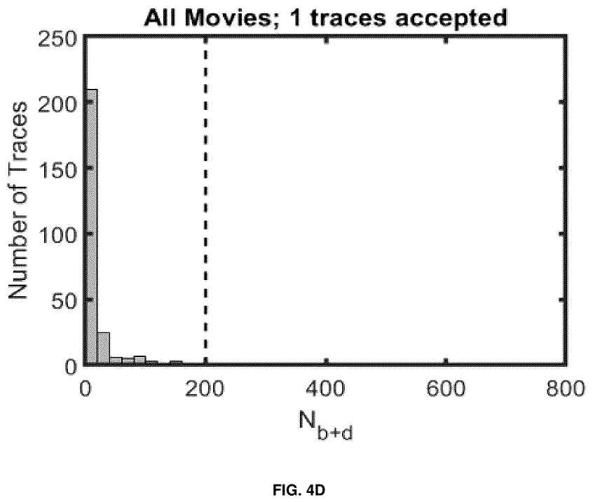

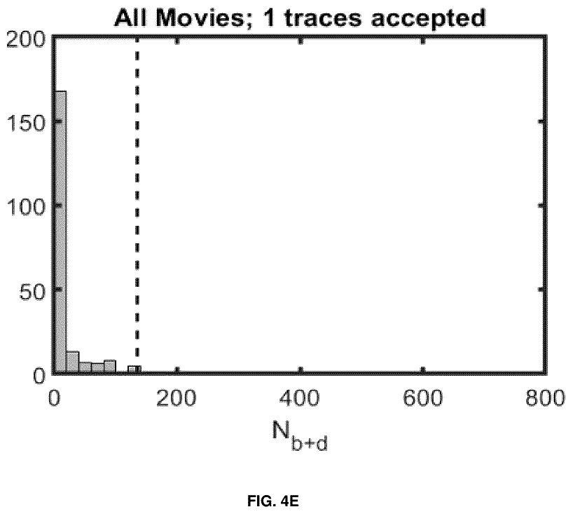

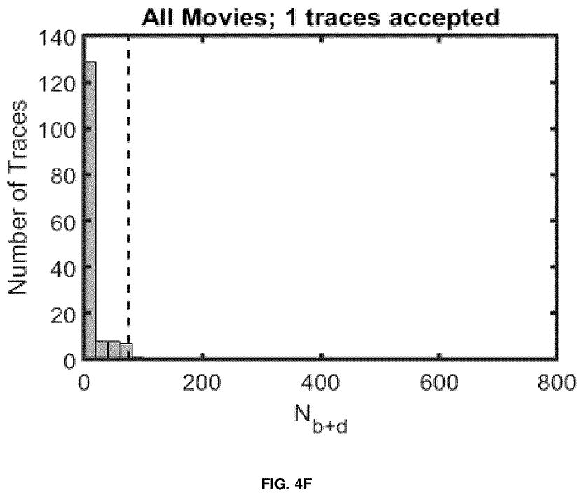

During the development of embodiments of the technology described herein, further experiments were conducted to test use of the F-gen V4 fluorogenic query probe for detecting the T790M target analyte. FIG. 4A-4C show data collected from experiments in which a sample comprising 100 fM of the T790M target analyte in IB/OSS comprising 10% w/v formamide was analyzed with 500 nM of the F-gen V4 query probe at 28° C. The laser power was 60 mW and 2 fields of view were imaged (TIRF 80 nm pd) with a 250-millisecond exposure time. Control experiments were performed under the same conditions except samples were prepared without the T790M target analyte (FIG. 4D-4F). These data indicated that the peak value of the N_{b+d} distribution decreased as a function of the time of data collected (2.5, 5, or 10 minutes of video collected as shown in FIG. 4A, FIG. 4B, and FIG. 4C, respectively). However, the number of accepted traces obtained from each analysis were comparable for the three different data collection times. One false positive was detected in the control experiment data (FIG. 4D-4F). These data indicate that using increased concentrations of a fluorogenic probe in SiMREPS assays allows the data collection time to be shortened without introducing high background and/or false positives.

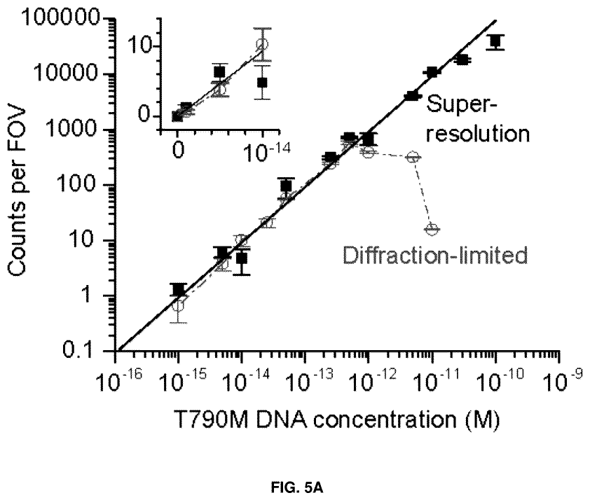

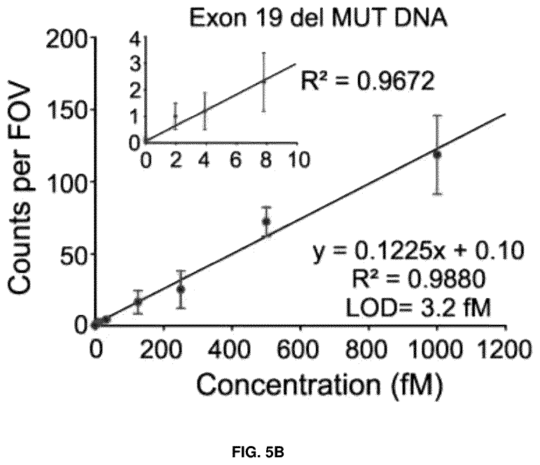

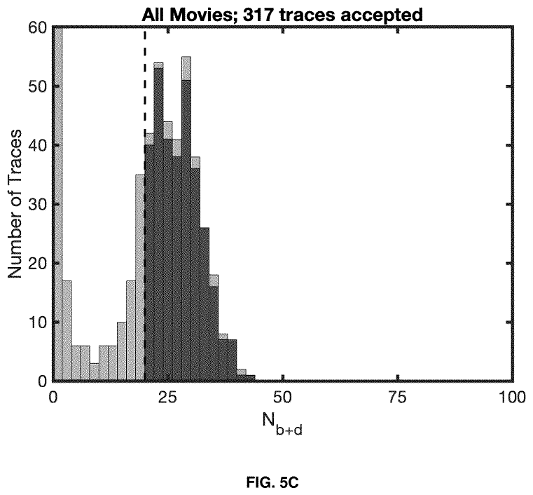

FIG. 5A-FIG. 5C show the results of experiments comparing conventional SiMREPS, an intramolecular SiMREPS technology (Khanna (2021) “Rapid kinetic fingerprinting of single nucleic acid molecules by a FRET-based dynamic biosensor” Biosensors and Bioelectronics 190: 113433, incorporated herein by reference), and the fluorogenic probe SiMREPS as described herein. FIG. 5A shows data from conventional SiMREPS in which a query probe comprising a fluorogenic moiety was used at 25 nM to detect a nucleic acid analyte present in the sample at 100 fM. 10 minutes of video were recorded, and approximately 100 counts of probe association with the target analyte were acceptable according to the quality and threshold criteria. FIG. 5B shows data from intramolecular SiMREPS in which a query probe comprising a Cy3 and Alexa Fluor 647 fluorescent moieties was used at 10-50 nM to detect a nucleic acid analyte present in the sample at 100 fM. 1-15 minutes of video were recorded, and approximately 15 counts of probe association with the target analyte were acceptable according to the quality and threshold criteria. FIG. 5C shows data from fluorogenic probe SiMREPS in which a fluorogenic probe comprising Cy5 and BHQ2 was used at 500 nM to detect a nucleic acid analyte present at 100 fM. 30 seconds of video were recorded, and approximately 160 counts of probe association with the target analyte were acceptable according to the quality and threshold criteria.

As indicated by the data in FIG. 5A-FIG. 5C, the number of accepted traces for detecting 100 fM of the nucleic acid target is higher when using the fluorogenic probe compared to conventional SiMREPS. Thus, these data indicate that limit of detection provided by the fluorogenic probe SiMREPS is lower. Further, the length of video needed to distinguish between true targets and nonspecific binding when using the fluorogenic probe is lower than conventional SiMREPS and comparable to intramolecular SiMREPS, but fluorogenic probe SiMREPS provides a much higher detection sensitivity than both conventional and intramolecular SiMREPS. This arises in part because, in some embodiments of intramolecular SiMREPS, the intramolecular SiMREPS probes sometimes undergo irreversible photobleaching and can thus only be imaged for approximately 30 to seconds. However, fluorogenic SiMREPS can be used to provide binding kinetics that are similar to intramolecular SiMREPS, but fluoregenic SiMREPS probes can be imaged for longer times per field of view (e.g., for 10 minutes or more).

### Example 2—Multiplexed SiMREPS

Simultaneous monitoring of multiple markers of the biological status of an organism is important for diagnosis. In particular, the accuracy, speed, and throughput of diagnostics can be improved by simultaneously detecting, quantifying, identifying, or otherwise characterizing (e.g., with respect to presence, absence, amount, concentration, state) multiple target analytes, some of which may be present in low concentrations. Thus, assays for multiple diagnostic biomarkers would improve clinical diagnostics and point-of-care testing.

For example, proteins and cell-free nucleic acids (e.g., circulating tumor DNA (ctDNA) and non-coding RNAs (e.g., miRNAs); see, e.g., Geekiyanage (2020) “Extracellular microRNAs in human circulation are associated with miRISC complexes that are accessible to anti-AGO2 antibody and can bind target mimic oligonucleotides” PNAS 117 (39): 24213-23, incorporated herein by reference; and Ns (2018) “Circulating cell-free nucleic acids: characteristics and applications” European Journal of Human Genetics 26: 937-45, incorporated herein by reference; see especially Ns at Tables 1 and 2, incorporated herein by reference) that circulate in biofluids (e.g., urine and blood) have emerged as potential cancer biomarkers. However, current technologies limit the simultaneous and direct detection of both protein and nucleic acids in biofluids because of the association of cell-free nucleic acids—especially miRNAs—with protective macromolecular complexes, including extracellular vehicles (EVs), proteins such as Argonaute 2 (AGO2), or high-density lipoproteins; and because suitable amplification techniques for proteins and short nucleic acids are in limited use or are not available.

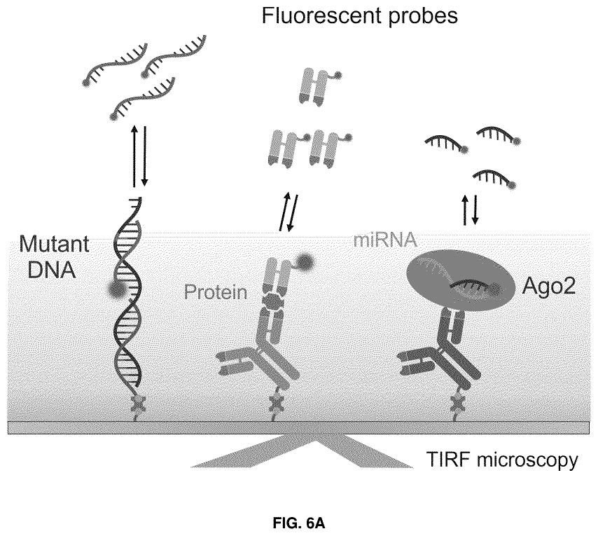

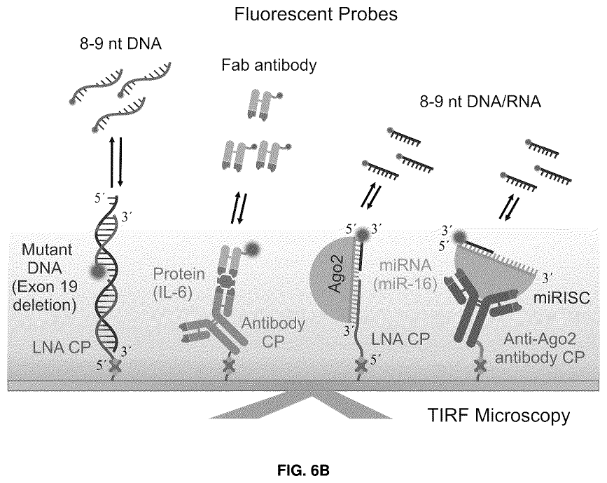

The technology described herein provides a SiMREPS technology for highly specific, sensitive, multiplexed (e.g., parallel) detection and digital counting of protein and small nucleic acids (e.g., miRNA and ctDNA) molecules in biofluids. For example, in some embodiments, the technology comprises use of a high-affinity antibody to stably surface capture a protein antigen, an anti-Ago2 antibody to stably surface capture miRNA loaded into Ago2 (i.e., a miRISC complex; see, e.g., Geekiyanage (2020) “Extracellular microRNAs in human circulation are associated with miRISC complexes that are accessible to anti-AGO2 antibody and can bind target mimic oligonucleotides” PNAS 117 (39): 24213-23, incorporated herein by reference), and/or a DNA capture probe (e.g., a locked nucleic acid (LNA)-modified DNA) to stably surface capture mutant DNAs related to different human diseases. In some embodiments, analytes are captured directly from human or human-derived samples (e.g., HeLa cell extracts). In some embodiments, an LNA-modified DNA is used to capture miRISC. See, e.g., FIG. 6A and FIG. 6B.

During the development of embodiments of the technology described herein, an exemplary single molecule kinetic fingerprinting approach for multiplex detection of diverse biomarkers was designed and tested (FIG. 6B). In these experiments, a nucleic acid target analyte comprising a sequence of EGFR exon 19 deletion mutant DNA (COSM6225) was surface captured using LNA-modified DNA capture probe and imaged by a Cy5-labeled 8 nt DNA fluorescent probe under an objective-TIRF microscope. IL-6 antigen was surface captured using an IL-6 antibody and imaged by a Cy5-labeled Fab fluorescent probe. miR-16 was detected as a miRISC complex captured on the surface either using an anti-Ago2 antibody or LNA-modified DNA capture probe and imaged by seed specific Cy5-labeled 8-nt DNA fluorescent probe. Experiments conducted during the development of embodiments of the technology indicated that the 3′ end of the target miRNA analyte (approximately nucleotides 1 to 8 at the 3′ end) is accessible for query probe access when the miRNA is bound to Argonaute.

During the experiments, a high affinity IL-6 antibody was used as a capture probe for IL-6, and a low affinity Fab (K_{D}=10-600 nM) was used as a query probe for IL-6. A high affinity anti-Ago2 antibody was used as a capture probe for miRISC complex (comprising miR-16) or a LNA-modified capture probe was used as a capture probe for miR-16, and a miR-specific query probe was used for miR-16. The miR-16 capture probe had a sequence of C+GC+CA+AT+AT+TT (SEQ ID NO: 7), where “+” indicates a LNA linkage. The miR-16 capture probe was modified at the 5′ end by a biotin-TEG. The miR-16 specific query probe had a sequence TGCTGCTA (SEQ ID NO: 8), was labeled at the 5′ end by Cy5, and had a Tₘ of 22.7° C. in 2×PBS. The EGFR exon 19 deletion mutant DNA target nucleic acid analyte had a sequence of TTC CCG TCG CTA TCA AGA CAT CTA GGA C (SEQ ID NO: 9). A capture probe for the EGFR exon 19 deletion mutant DNA target nucleic acid analyte had a sequence of +AG+CG+ACG+GG+AA (SEQ ID NO: 10), where “+” indicates a LNA linkage. The EGFR exon 19 deletion mutant DNA target nucleic acid analyte capture probe was modified on the 3′ end by a biotin-TEG. The query probe for the EGFR mutant target nucleic acid analyte had a sequence of AT GTC TTG (SEQ ID NO: 11), was labeled at the 5′ end by Cy5, and had a Tₘ of 14.5° C. in 2×PBS.

During the development of embodiments of the technology described herein, experiments were conducted in which SiMREPS used an objective-TIRF microscope for imaging transiently bound fluorescent probes with the target analytes. For protein (e.g., IL-6), mutant DNA (e.g., EGFR exon 19 deletion mutant DNA (COSM6225)), and miRNA (e.g., miR-16 as miRISC complex) detection experiments, sample cells (e.g., made from cut pipette tips) were attached to a passivated glass coverslip comprising biotin-PEG and m-PEG (biotin-PEG: m-PEG=1:100). The sample cells were first treated with 40 μL of 1 mg/mL streptavidin in T50 (10 mM Tris-HCl pH 8.0, 50 mM NaCl) for 15 minutes and each cell was washed 3 times using T50 after removing unbound streptavidin. Next, the capture probes (e.g., 80 μL of biotin-IL-6 antibody, biotin-LNA modified DNA comprising a sequence specific to a part of the mutant DNA, biotin-anti-Ago2 antibody, or biotin-LNA modified DNA specific to a part of the miR-16 RISC complex) were added at 100 nM final concentration in 1×PBS (10 mM Na₂HPO₄, 1.8 mM KH₂PO₄, pH 7.4, 137 mM NaCl, 2.7 mM KCl) containing 10 mg/mL BSA were added in each cell either individually or in combination for 30 minutes. After removal of unbound capture probes on the surface, each cell was washed 3 times using 1×PBS. The target solution was prepared by spiking IL-6 antigen and EGFR exon 19 deletion mutant DNA into 0.01% HeLa cell extract to provide a final concentration of 500 fM and 10 fM for IL-6 and EGFR mutant DNA, respectively. 100 μL of the target solution was introduced into each cell for surface capture of single or multiple analytes based on the capture probe(s) tethered on the surface. After 1 hour of incubation the target solution was removed from each cell and washed 3 times using 2×PBS (20 mM Na₂HPO₄, 3.6 mM KH₂PO₄, pH 7.4, 274 mM NaCl, 5.4 mM KCl). The specific target(s) were imaged using 200 μL of fluorescent probe(s) at a 50 nM final concentration in 2×PBS containing 1 mg/mL BSA, 1% tween 20, and oxygen scavenger system (OSS) at 29° C.

SiMREPS experiments were performed using an Olympus IX-81 objective-type TIRF microscope equipped with a 60× oil-immersion objective (APON 60XOTIRF, 1.49 NA) with Cell TIRF and z-drift control modules. Fluorescent probes were excited by a 640-nm red laser (Coherent CUBE 640-100C, 100 mW) in TIRF mode with a theoretical penetration depth of approximately 100 nm with an incident light intensity of approximately 100 W/cm². Fluorescence emission was detected using an EMCCD (electron-multiplying charge-coupled device: Andor IXon 897, or Photometrics Evolve) with an exposure time of 300 ms for 10 minutes, after passing through a dichroic mirror and emission filter (Chroma, ZT640rdc-UF2 and ET655LP-TRF). An objective heater (Bioptechs) was used to raise the observation temperature to 29° C.

Analysis was performed with two MATLAB programs called SiMREPS analysis suite (SAS) and SiMREPS optimizer. SAS uses intensity averaging to identify spots of high average fluorescence intensity within each field of view, generates intensity-versus-time traces from those spots, fits the intensity-versus-time traces using a two-state HMM algorithm to generate idealized (noise-less) intensity-versus-time traces to identify transitions between high and low intensity states, and applies kinetic filtering to distinguish target-bound molecule traces from non-target traces and background. Table 2 provides the acquisition parameters and default kinetic filtering criteria for SiMREPS for detecting IL-6, EGFR exon 19 deletion mutant DNA, and miR-16 based on metrics including the N_{b+d}, dwell times in the high- and low-intensity states, S/N, and signal intensity, which provide criteria for identifying the target, maximizing counts of genuine molecules, and minimizing false positives.

TABLE 2
Acquisition and filtering criteria

| | |
|---|---|
| Movie frames start-to-end | 1-2000 |
| Exposure time per frame (s) | 0.3 |
| Acquisition time (min) | 10 |
| Intensity threshold per trace | 1500 |
| S/N threshold per event | 2.5 |
| S/N threshold per trace | 2.5 |
| Minimum N_{b+d} | 15 |
| Maximum N_{b+d} | Inf |
| Minimum τ_{on, median} (s) | 0.3 |
| Maximum τ_{on, median} (s) | 60 |
| Minimum τ_{off, median} (s) | 0.3 |
| Maximum τ_{off, median} (s) | 60 |
| Maximum τ_{on, event} (s) | 90 |
| Maximum τ_{off, event} (s) | 90 |

The SiMREPS optimizer uses data from experiments with and without the target as positive and negative training sets, respectively, for a Monte Carlo optimization that outputs an initial list of optimized filtering parameters that can be further refined by hand for higher specificity and sensitivity. Essentially, the SiMREPS optimizer extracts the kinetic parameters for accurately detecting a distinct subset of kinetic fingerprinting (e.g., for a specific target) in a mixture using data from experiments with two different targets as positive and negative training sets.

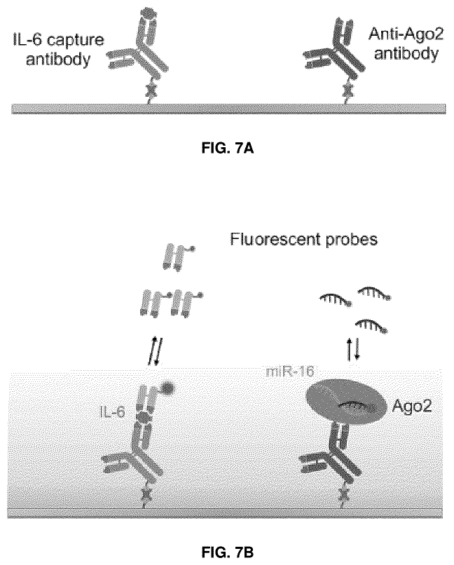

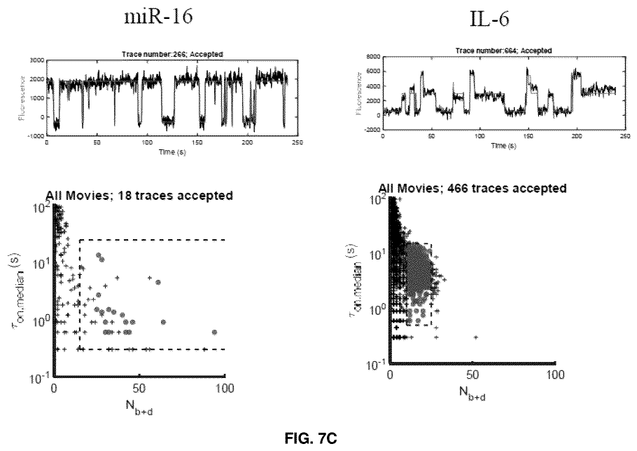

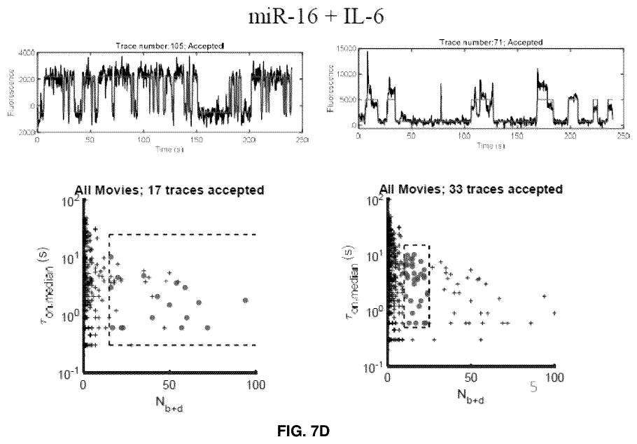

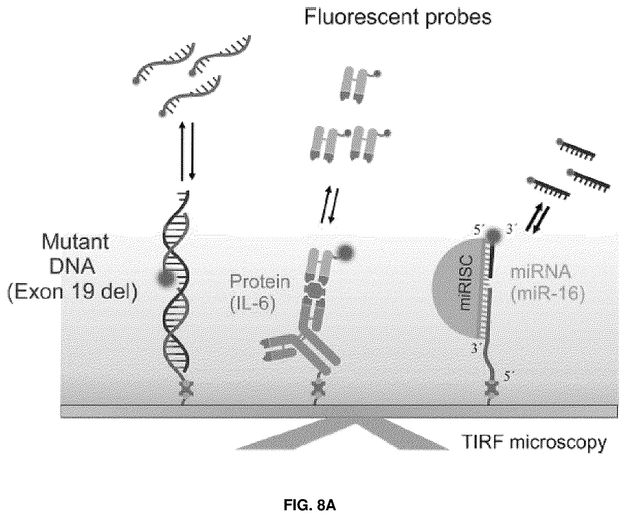

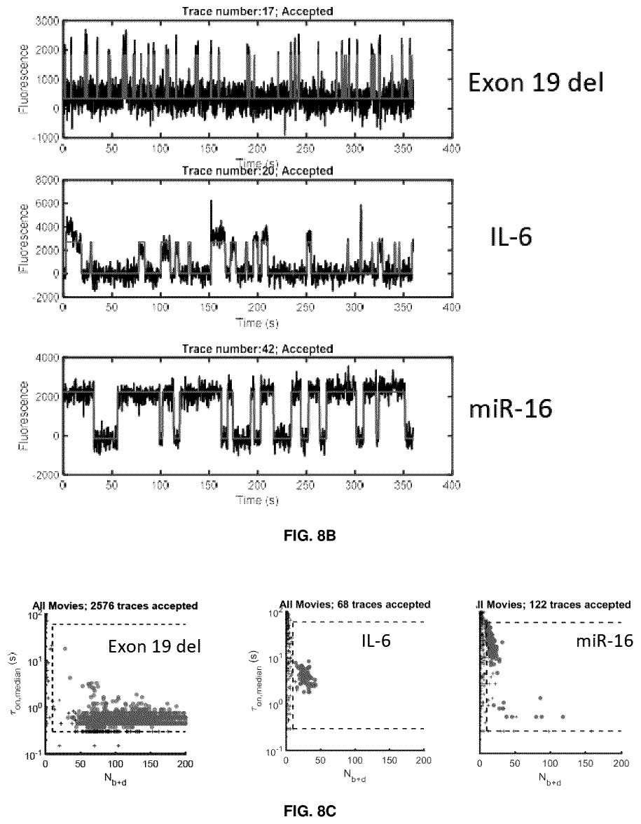

Data collected during these experiments is shown in FIG. 7A-7D; and in FIG. 8A-FIG. 8B. FIG. 7A to FIG. 7D show the results of a multiplex SiMREPS experiment in which IL-6 and miR-16 were detected in the same sample. An anti-IL-6 antibody and an anti-Ago2 antibody were used as capture probes (FIG. 7A); and fluorescently labeled query probes discussed above were used to produce kinetic data for IL-6 and miR-16 (FIG. 7B). FIG. 7C shows data collected from experiments in which each of miR-16 and IL-6 were detected alone (e.g., non-multiplexed) and FIG. 7D shows data collected from a multiplex experiment in which IL-6 and miR-16 were present in the same sample. As shown in FIG. 7C by the boxed dots, the IL-6 and miR-16 analytes produced distinct signals (kinetic fingerprints) on the plots of τ_{on, median} versus N_{b+d}. These distinct signals (kinetic fingerprints) were used as training data to detect both IL-6 and miR-16 in the same sample in multiplex SiMREPS as shown in the plots of τ_{on, median} versus N_{b+d} in FIG. 7D.

FIG. 8A is a schematic drawing showing a multiplex experiment in which IL-6, miR-16, and EGFR exon 19 deletion mutant DNA are detected in the same sample and, in some embodiments, in the same field of view. FIG. 8B and FIG. 8C show data from experiments in which a single target was assayed with a single fluorescent probe to show that probe designs for these three analytes generate distinct kinetic fingerprints that could be distinguished in a multiplex SiMREPS assay. An anti-IL-6 antibody was used as a capture probe for IL-6; and LNA capture probes were used to capture the EGRF mutant DNA and miR-16 microRNA. The capture probes are described above. See FIG. 8A. FIG. 8B shows kinetic data collected from the SiMREPS experiments to assay IL-6, miR-16, and the EGFR deletion mutant DNA. FIG. 8C shows plots of τ_{on, median} versus N_{b+d} derived from the kinetic data of FIG. 8B. As shown in FIG. 8C, each of the three analytes produced a distinct signal in the plots of τ_{on, median} versus N_{b+d}, from which the three analytes could be distinguished.

The data collected during these multiplex SiMREPS experiments indicated that protein and nucleic acid analytes are distinguishable and detectable using the multiplex SiMREPS. Additional analytes that are tested in experiments include, e.g., IL-6, miR-141, and EGFR exon 19 deletion mutant; multiple HPV cfDNA sequences (e.g., from urine); and IL-6, miR-150, and a chimeric antigen receptor T-cell sequence.

A multiplexed assay is used to detect, quantify, identify, or otherwise characterize (e.g., with respect to presence, absence, amount, concentration, state) the multiple target analytes. In some embodiments, the technology comprises optimizing and/or tuning the relative thermodynamic stabilities of multiple fluorescent probes specific for the multiple target analytes (e.g., the protein antigen, seed region of miRISC, and mutant DNA) to provide appropriate kinetics for SiMREPS assays.

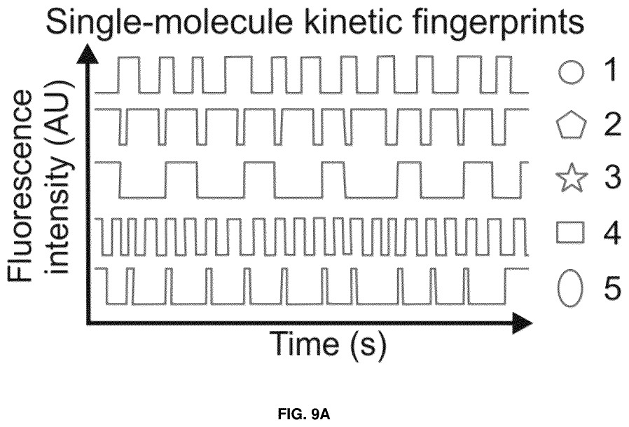

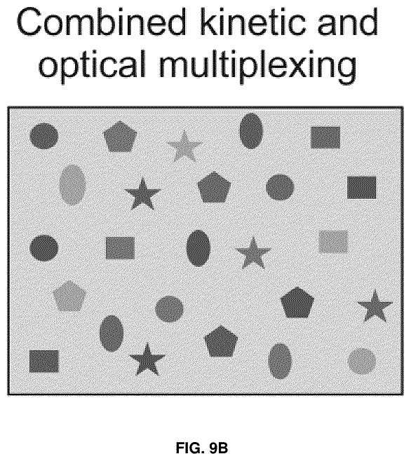

In some embodiments, this kinetic multiplexing generates distinct digital fingerprints for each target analyte in a single field of view of a fluorescence microscope. In some embodiments, Poisson statistical treatment finds use in distinguishing the digital fingerprints from each other. For example, as shown in FIG. 9A, in some embodiments, different analytes (e.g., as shown by the circle, pentagon, star, rectangle, and oval) have different SiMREPS time-dependent intensity signals that are used to detect, quantify, identify, or otherwise characterize (e.g., with respect to presence, absence, amount, concentration, state) the multiple target analytes. In some embodiments, the time-dependent intensity signals have differences in the fluorescence intensities of the transitions, differences in the dwell times in the unbound state, differences in the dwell times in the bound state, differences in the kinetic dissociation constant, and/or differences in the kinetic association constant.

In some embodiments, multiplexed SiMREPS combines other signal types with the kinetic fingerprint signal. For instance, in some embodiments, kinetic fingerprints are combined with other signals of distinguishing single molecule trajectories from each other—e.g., signal intensity, fluorophore color (see, e.g., FIG. 9B), and other patterns of distinguishing features to provide rapid and massively parallel one-pot detection of large numbers of diverse biomarkers in clinical sample. In some embodiments, the technology uses deep learning and artificial intelligence algorithms to maximize the accuracy of assigning signals to biomarkers.

### Example 3—Single-Color Multiplexed SiMREPS

During the development of embodiments of the technology described herein, experiments were conducted to test multiplex detection of multiple protein analytes using query probes comprising the same fluorescent label. During these experiments, the protein analytes were differentiated from each other using differences in the kinetic traces (time-dependent changes in signal) produced by query probes specific for each protein analyte.

In particular, A 3-plex measurement of the protein targets beta-nerve growth factor (beta-NGF), interleukin-10 (IL-10), and interleukin-11 (IL-11) was performed as follows. Sample wells fixed to glass coverslips functionalized with a 1:100 mixture of biotin-PEG and mPEG (PEG=polyethylene glycol, molecular weight approximately 5000 Daltons) were hydrated for 10 min with 1× phosphate buffered saline (PBS), pH 7.4, and then incubated for 10 minutes with a 1 mg/mL streptavidin solution. The streptavidin solution was removed, and the sample wells were washed four times with 1×PBS. The final wash was removed, and a mixture containing three biotinylated capture antibodies in 1×PBS was added. Specifically, the mixture contained 50 nanomolar of a capture antibody for beta-NGF and 25 nanomolar each of capture antibodies for IL-10 and IL-11. The capture antibody solution was incubated in the wells for 30 minutes, and then it was removed. The wells were washed four times with 1×PBS. Next, 15-μL solutions containing 32 or 0 pg/mL of beta-NGF, 32 or 0 pg/mL of IL-11, or 64 or 0 pg/mL of IL-10—including samples containing all three antigens or none of the antigens—in 10 mg/mL BSA were added to each sample well and incubated for 1 hour at 25° C. to capture the antigens on the surface.

Next, to each well was added 45 uL of an imaging solution containing a PCD/PCA/Trolox oxygen scavenger and triplet-state quencher system in 1×Tris-buffered saline (TBS), pH 8.0, and query probes specific for the three analytes beta-NGF, IL-10, and IL-11. In particular, each query probe in this experiment comprised an Alexa Fluor 647-labeled detection antibody specific for beta-NGF, IL-10, or IL-11. In the final mixture, the concentrations of the beta-NGF, IL-11, and IL-10 query probes (detection antibodies) were 25, 25, and 50 nanomolar, respectively. The final concentrations of the beta-NGF, IL-11, and IL-10 analytes were 8 or 0 pg/mL, 8 or 0 pg/mL, and 16 or 0 pg/mL, respectively. After an additional 30 minute incubation period, the samples were imaged by total internal reflection fluorescence (TIRF) microscopy with a 60× objective and excitation at 640 nanometers, using a 5-minute data acquisition time per field of view and a 2-Hz frame rate. The TIRF microscopy measurement had a single detection band for emission from Alexa Fluor 647, the only fluorophore used in this measurement. The movies were analyzed for single-molecule kinetic fingerprints using modifications of methods described in Chatterjee (2020) “Direct kinetic fingerprinting and digital counting of single protein molecules” Proc. Natl. Acad. Sci. USA 117 (37): 22815-22, which is incorporated herein by reference.

Using an automated optimization program in Matlab, three different sets of kinetic filtering parameters were found that maximized the number of accepted single-molecule traces (that is, traces classified as positive detection events) in the presence of each antigen while rejecting the vast majority of single-molecule traces in the absence of that antigen but in the presence of the other antigens. For example, beta-NGF was detected using a set of kinetic filtering parameters that accept the majority of single-molecule traces in movies from sample wells containing 8 pg/mL beta-NGF while rejecting the majority of single-molecule traces in movies from sample wells containing 8 pg/mL IL-11 or 16 pg/mL IL-10 but not containing beta-NGF. Note that this also provides the ability to distinguish signals arising from beta-NGF, IL-10, and IL-11, even when all are present in the same sample and the same microscopic field of view. Example kinetic filtering parameters for this assay are shown in Table 3.

TABLE 3
Kinetic filtering parameters used in 3-plex protein measurement

| Target: | Beta-NGF | IL-10 | IL-11 |
|---|---|---|---|
| Intensity Threshold | 1000 | 1000 | 1000 |
| Max Baseline Intensity | 4500 | 3200 | 10000 |
| S/N Threshold (Event) | 3 | 3 | 3 |
| S/N Threshold (Trace) | 6.1 | 2.1 | 0 |
| Minimum N_{b+d} | 2 | 5 | 27 |
| Maximum N_{b+d} | 57 | 17 | 60 |
| Minimum Tau(Bound, Median) (s) | 0 | 2.5 | 1.5 |
| Maximum Tau(Bound, Median) (s) | 1.5 | 41 | 26 |
| Minimum Tau(Unbound, Median) (s) | 3.5 | 7.5 | 3 |
| Maximum Tau(Unbound, Median) (s) | 57 | 154 | 70 |
| Maximum Individual Tau(Bound) (s) | 5.5 | 70 | Inf |
| Maximum Individual Tau(Unbound) (s) | Inf | Inf | Inf |
| Maximum Tau(Bound) C.V. | Inf | Inf | 3.4 |
| Maximum Tau(Unbound) C.V. | Inf | 4.4 | Inf |

The primary criteria used to distinguish between the kinetic fingerprints of the three targets are; minimum and maximum values of N_{b+d} (number of binding and dissociation events per single-molecule trace within the 5-minute observation period); minimum and maximum values of the median of the bound-state dwell time (Tau(Bound,Median)); and the minimum and maximum values of the median of the unbound-state dwell time (Tau(Unbound,Median)). For example, beta-NGF has a minimum and maximum Tau(Bound,Median) of 0 and 1.5 seconds, respectively, a range which does not overlap with the ranges for IL-10 or IL-11. Similarly, IL-11 has a minimum and maximum N_{b+d} value of 27 and 60, respectively, which does not overlap with the range of 5-17 for IL-10. The detailed kinetic information extracted from the single-molecule traces thus provides several options by which different antibody-target interactions can be distinguished, even if they are detected using the same fluorescent label. In addition to the above metrics, other statistics can be used to accept or reject signals as evidence of a given target, including: the coefficient of variation (C.V.) of all the bound-state or unbound-state dwell times in each single-molecule trace (Tau(Bound) C.V. and Tau(Unbound) C.V.); maximum observed dwell times in the bound state or unbound state within each single-molecule trace (Maximum Individual Tau(Bound) and Maximum Individual Tau(Unbound)); the minimum allowable signal-to-noise ratio for individual binding events (S/N Threshold (Event)) or for an entire single-molecule trace (S/N Threshold (Trace)); and the maximum allowable intensity of the baseline signal in a single-molecule trace (Max Baseline Intensity). In general, however, N_{b+d} and Tau metrics were more useful for distinguishing the protein targets in this assay.

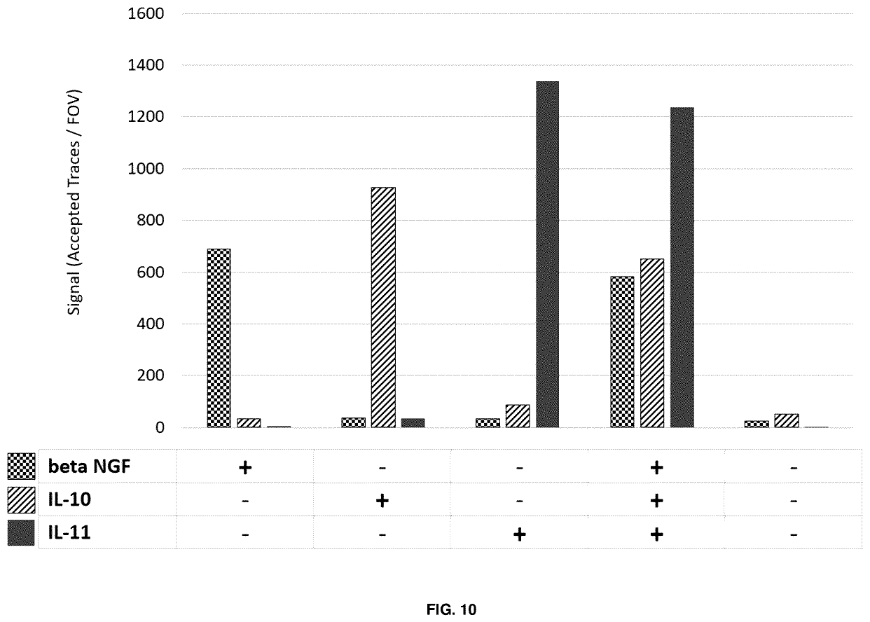

Using this approach, in a single 5-minute SiMREPS measurement per sample well, kinetic information was used to detect and quantify each of the three targets with low cross-talk from the other two targets (FIG. 10). Sensitivity for each analyte was between 50 and 100% of the sensitivity in the corresponding single-plex assay containing only a single capture and detection antibody pair.

### Example 4—Single-Color Multiplexed SiMREPS with Aptamer and Antibody Query Probes

During the development of embodiments of the technology described herein, a second set of experiments was conducted to test multiplex detection of multiple protein analytes using query probes comprising the same fluorescent label. During these experiments, the protein analytes were differentiated from each other using differences in the kinetic traces (time-dependent changes in signal) produced by query probes specific for each protein analyte. In this set of experiments, both antibody and nucleic acid (aptamer) query probes were used.

In particular, A 3-plex measurement of the protein targets interleukin-11 (IL-11), interleukin-8 (IL-8), and interleukin-6 (IL-6) was performed as follows. Sample wells fixed to glass coverslips functionalized with a 1:100 mixture of biotin-PEG and mPEG (PEG=polyethylene glycol, molecular weight approximately 5000 Daltons) were hydrated for 10 min with 1× phosphate buffered saline (PBS), pH 7.4, and then incubated for 10 minutes with a 1 mg/mL streptavidin solution. The streptavidin solution was removed, and the sample wells were washed four times with 1×PBS. The final wash was removed, and a mixture containing three biotinylated capture antibodies in 1×PBS was added. Specifically, the mixture contained 10 nanomolar each of capture antibodies for IL-11, IL-8, and IL-6. The capture antibody solution was incubated in the wells for 30 minutes, and then removed. The wells were washed four times with 1×PBS. Next, 15-μL solutions containing 64 or 0 pg/mL of IL-11, 64 or 0 pg/mL of IL-6, or 32 or 0 pg/mL of IL-8—including samples containing all three antigens or none of the antigens—in 10 mg/mL BSA were added to each sample well and incubated for 30 min at 25° C. to capture the antigens on the surface.

Next, to each well was added 45 μL of an imaging solution containing a PCD/PCA/Trolox oxygen scavenger and triplet-state quencher system in 1×Tris-buffered saline (TBS), pH 8.0, and query probes specific for the three analytes IL-11, IL-6, and IL-8. In particular, this experiment used query probes that comprised an Alexa Fluor 647-labeled detection antibody against IL-11, an Alexa Fluor 647-labeled detection antibody against IL-6, or an Alexa Fluor 647-labeled detection aptamer against IL-8. In the final mixture, the concentrations of the IL-11 and IL-6 detection antibodies were 10 and 25 nM, respectively, and the concentration of the IL-8 aptamer was 40 nM. The final concentrations of the IL-11, IL-6, and IL-8 analytes were 16 or 0 pg/mL, 16 or 0 pg/mL, and 8 or 0 pg/mL, respectively. After an additional 30 minute incubation period, the samples were imaged by total internal reflection fluorescence (TIRF) microscopy with a 60× objective and excitation at 640 nanometers, using a 5-minute data acquisition time per field of view and a 2-Hz frame rate. The TIRF microscopy measurement had a single detection band for emission from Alexa Fluor 647, the only fluorophore used in this measurement. The movies were analyzed for single-molecule kinetic fingerprints using modifications of methods described in Chatterjee (2020) “Direct kinetic fingerprinting and digital counting of single protein molecules” Proc. Natl. Acad. Sci. USA 117 (37): 22815-22, which is incorporated herein by reference.

Using an automated optimization program in Matlab, three different sets of kinetic filtering parameters were found that maximized the number of accepted single-molecule traces (that is, traces classified as positive detection events) in the presence of each antigen while rejecting the vast majority of single-molecule traces in the absence of that antigen but in the presence of the other antigens. Example kinetic filtering parameters for this assay are shown in Table 4.

TABLE 4
Kinetic filtering parameters used in 3-plex protein measurement

| Target: | IL-11 | IL-8 | IL-6 |
|---|---|---|---|
| Intensity Threshold | 1000 | 1000 | 1000 |
| Max Baseline Intensity | 1700 | 10500 | 2200 |
| S/N Threshold (Event) | 3 | 3 | 3 |
| S/N Threshold (Trace) | 6.7 | 3.1 | 5.4 |
| Minimum N_{b+d} | 4 | 19 | 3 |
| Maximum N_{b+d} | 104 | 137 | 14 |
| Minimum Tau(Bound, Median) (s) | 1 | 0 | 8 |
| Maximum Tau(Bound, Median) (s) | 4.5 | 2 | 56.5 |
| Minimum Tau(Unbound, Median) (s) | 8.5 | 6.5 | 5.5 |
| Maximum Tau(Unbound, Median) (s) | 109.5 | 99 | 140.5 |
| Maximum Individual Tau(Bound) (s) | 20.5 | 6.5 | 98 |
| Maximum Individual Tau(Unbound) (s) | 155.5 | Inf | Inf |
| Maximum Tau(Bound) C.V. | Inf | Inf | Inf |
| Maximum Tau(Unbound) C.V. | Inf | Inf | Inf |

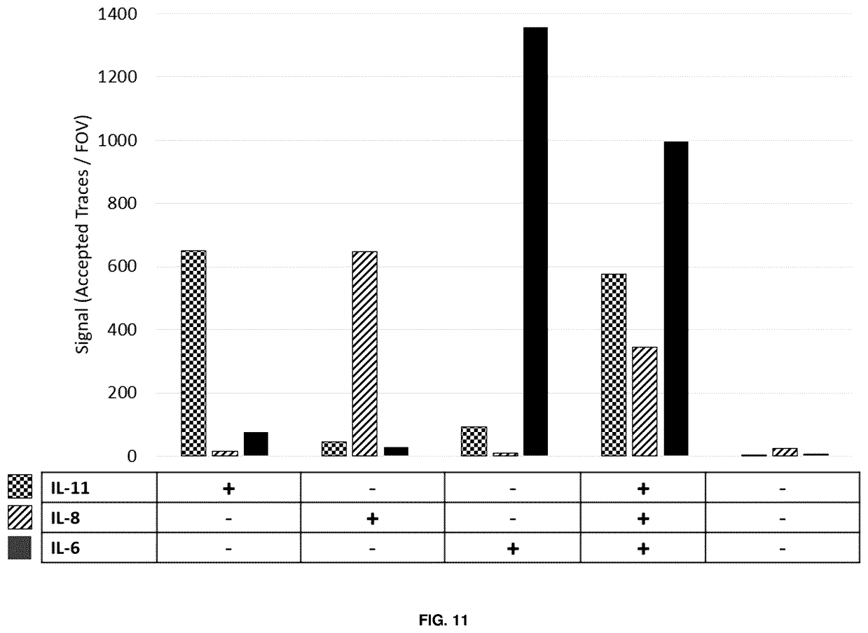

Using this approach, in a single 5-minute SiMREPS measurement per sample well, kinetic information was used to detect and quantify each of the three targets with low cross-talk from the other two targets (FIG. 11). Notably, to provide this low level of cross-talk, a higher concentration of the IL-8 detection aptamer (40 nM) was used than of the IL-11 detection antibody (10 nM) so that the kinetic fingerprints corresponding to IL-8 and IL-11 were distinguishable within the acquisition time of the experiment. This is because the IL-8 aptamer and IL-11 detection antibody have similar kinetic fingerprints if the two are present at the same concentration, making it difficult to distinguish them without prolonged data acquisition (e.g., tens of minutes). This illustrates the ability to modulate kinetic fingerprints by adjusting concentrations of individual query probes in the imaging solution to achieve facile multiplexing. Sensitivity for each analyte was between 15 and 53% of the sensitivity in the corresponding single-plex assay containing only a single capture and detection antibody pair.

### Example 5—Fluorogenic SiMREPS

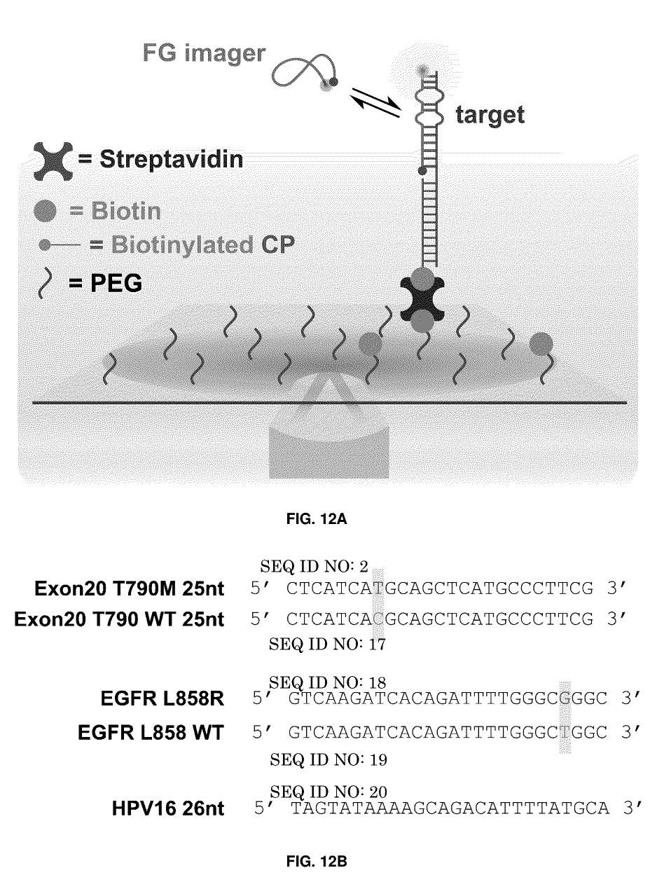

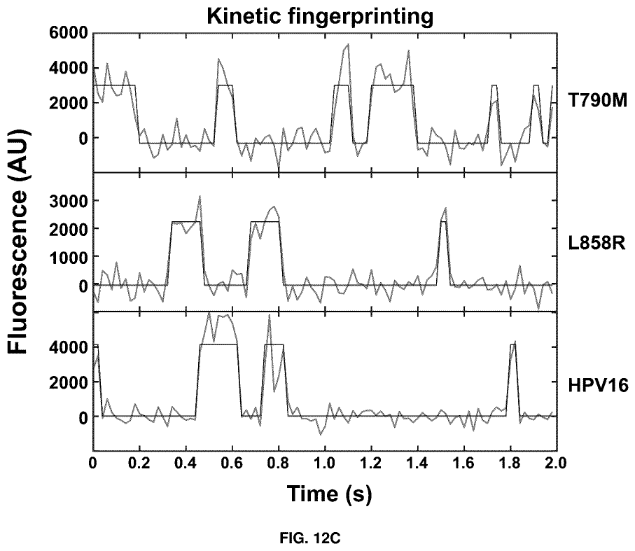

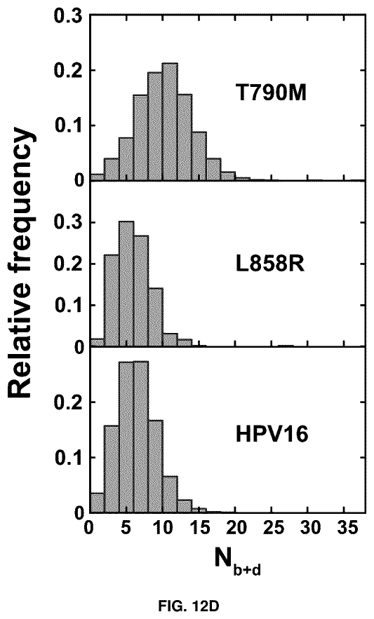

During the development of embodiments of the technology described herein, experiments were conducted to detect nucleic acids using fluorogenic SiMREPS. FIG. 12A shows a schematic of the experimental design for the fluorogenic SiMREPS (FG-SiMREPS) experiments. A glass coverslip was functionalized with polyethylene glycol-biotin, then coated with streptavidin via biotin-streptavidin interaction. A biotinylated nucleic acid was used as a capture probe. The capture probe (CP) was immobilized by interaction of the capture probe biotin and streptavidin. The capture probe binds nucleic acid biomarker target to the surface through base pairing. A dual-labeled nucleic acid imager comprising a green fluorophore at one end and a quencher at the other end was added at a micromolar concentration. The length and sequence of the fluorogenic imager (FG imager) was designed so that it was minimally or non-fluorescent while unbound or free in solution and emitted strong fluorescence when bound to a target on a TIRF-illuminated surface. FIG. 12B provides sequences of nucleic acid biomarker targets analyzed during the experiments: Exon20 T790M (SEQ ID NO: 12), Exon29 T790 WT (SEQ ID NO: 13), EGFR L858R (SEQ ID NO: 14), EGFR L858 WT (SEQ ID NO: 15), and HPV16 (SEQ ID NO: 16). Data were collected in experiments designed to detect the Exon20 T790M mutation and the EGFR L858R mutation and to distinguish single-nucleotide polymorphisms. Data indicated that the HPV16 target was detected by quantifying the HPV16 target relative to the substrate background signal. Kinetic fingerprinting data were acquired for each target. FIG. 12C. The data indicated that the binding kinetics of the fluorogenic imager probe were optimized to provide a sufficient number of transitions within a 2-s acquisition time. The transition data were recorded and used to distinguish mutant nucleic acids from nucleic acids having a wildtype signal and/or from background signals. In FIG. 12C, the green solid lines represent the raw intensity-time trajectories and the blue solid lines represent hidden Markov model (HMM) trajectories. The data were used to determine distributions of the number of binding and dissociation events (N_{b+d}) for detecting each target. FIG. 12D. The N_{b+d} distribution of a target signal was well separated from wildtype and background signals in all cases.

### Example 6—Multiplexed SiMREPS

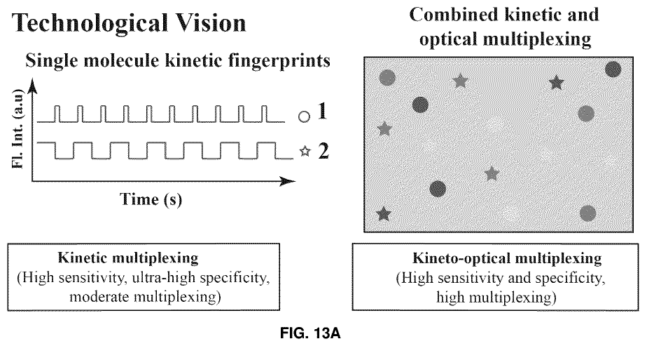

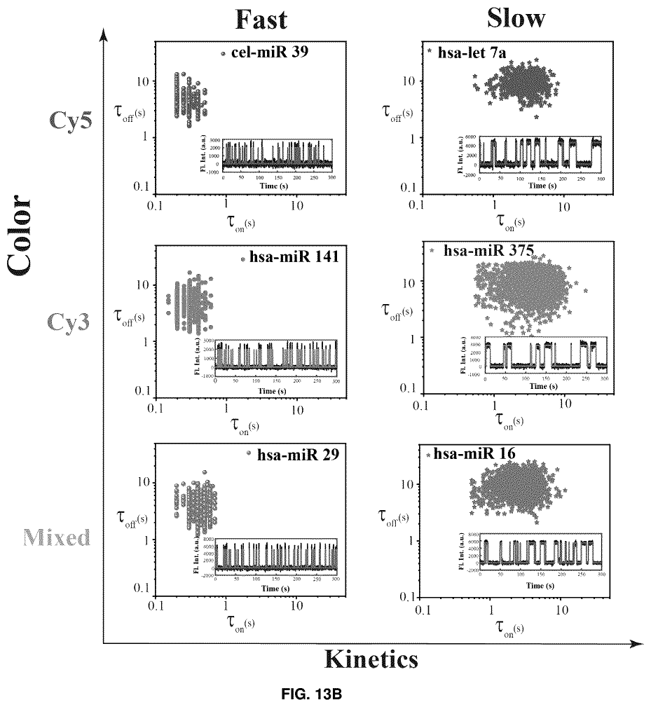

During the development of embodiments of the technology described herein, experiments were conducted to detect multiple analytes using multiplexed SiMREPS. FIG. 13A shows a schematic of multiplexed SiMREPS using kinetic fingerprinting and different colors. Using kinetic fingerprinting (“kinetic multiplexing”), at least two targets can be identified in a single field of view. In FIG. 13A, fast kinetics (relatively fast τₒₙ) is indicated by circle and slow kinetics (relatively slow τₒₙ) is indicated by star, where τₒₙ is the time of binding of the fluorescent probe with the target on a TIRF-illuminated surface. Further, combining multiple (e.g., three) different color channels with kinetic fingerprinting (“kineto-optical multiplexing”) enhances the multiplexing by another three fold (2 kinetic channels×3 color channels). In FIG. 13B, the cy5 channel is shown in red, the cy3 channel is shown in cyan, and the mixed channel of both cy3 and cy5 is shown in yellow-green. In the mixed channel, cy5-labeled nucleic acids and cy3-labeled nucleic acids provide fluorogenic probes and are mixed in equimolar amounts for a particular miRNA.

FIG. 13B shows data from experiments conducted to identify experimental and imaging conditions for use in detecting six different microRNAs (miRNAs). A pegylated glass coverslip was coated with streptavidin (1 mg/ml) via biotin-streptavidin interaction. A biotinylated nucleic acid capture probe (100 nM) was immobilized on the glass coverslip for binding miRNA targets through base pairing with the capture probes. The glass coverslip comprising immobilized capture probes was incubated with 5 pM of miRNA targets for 60 minutes. Short fluorescent DNA probes (8-10 nt) comprising either cy3 or cy5 fluorophore (50 nM) were added to the assay composition comprising OSS, and data were collected at room temperature for 5 minutes. The data collected indicated that the fluorescent probes transiently bound to the miRNA targets on the TIRF-illuminated surface and generated different kinetic patterns in distinct optical channels. τₒₙ vs τ_{off} plots were produced for the fields of view recorded for each experiment. FIG. 13B. Real target molecules were separated from blank by using threshold parameters as follows: N_{b+d}=20, τₒₙ (median bound time) greater than or equal to 0.2 s, and τ_{off} (median unbound time) greater than or equal to 0.2 s for all six miRNAs. Six miRNAs were channelized based on their τₒₙ values and fluorophore content. Fast channel miRNAs (i.e., cel-miR 39, hsa-miR 141, and hsa-miR29) had τₒₙ values of approximately 0.2 s to 1 s, and slow channel miRNAs (i.e., hsa-let 7a, hsa-miR 375, and hsa-miR 16) had τₒₙ values of approximately 1 s to 10 s. These data indicated that combining kinetic fingerprinting with different optical channels (cy5 in red (640 nm excitation), cy3 in cyan (561 nm excitation), mixed channel (cy3/cy5 alternatively) in dark yellow) provided an assay for multiplexed SiMREPS assay of multiple analytes simultaneously. Representative single molecule trace behaviors are shown in the insets of FIG. 13B. All experiments were performed using an Oxford Nanoimager (ONI) microscope.

### Example 7—Kinetic and Optical Multiplexing of miRNAs

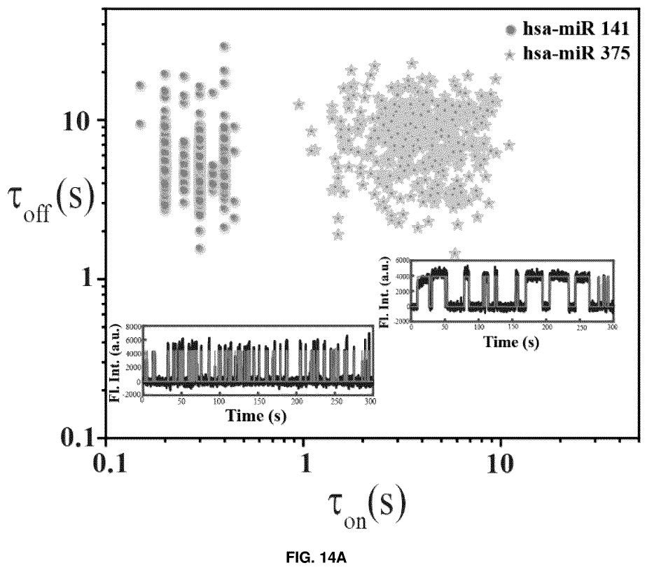

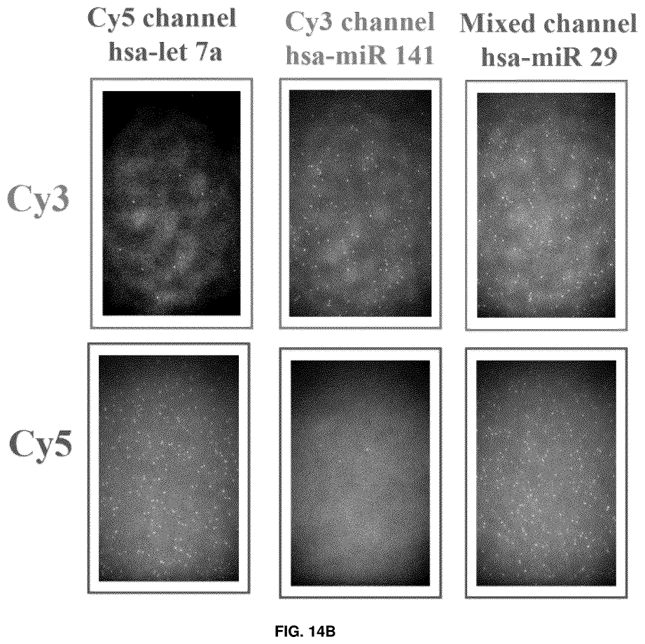

During the development of embodiments of the technology described herein, experiments were conducted to test kinetic and optical multiplexed SiMREPS detection of miRNA. Data were collected from experiments that used kinetic multiplexing to measure two miRNAs (i.e., hsa-miR 141 and hsa-miR 375) in a cy3 optical channel using kinetic fingerprinting. FIG. 14A. hsa-miR 141 has fast kinetics with <τₒₙ>=0.28±0.02 s and has-miR 375 has slow kinetics with <τₒₙ>=3.81±0.04 s. Capture probes (CPs) were used at 50 nM, miRNA targets were used at 5 pM, and fluorophores (FPs) were used at 50 nM. All other capturing conditions, imaging conditions, SiMREPS analysis conditions, and threshold parameters were the same as for the individual miRNA experiments as shown in FIG. 13B. A τₒₙ vs τ_{off} plot is shown for single field of view in FIG. 14A. Representative single molecule trace behaviors are shown in the insets. FIG. 14B shows the optical multiplexing of three miRNAs (i.e., hsa-let 7a in cy5 channel, hsa-miR 141 in cy3 channel, and hsa-miR 29 in mixed channel) using cy5 and cy3 separately in respective channels and alternatively in mixed channels. Capture probes (CPs) were used at 33.33 nM, miRNA targets were used at pM, and fluorophores (FPs) were used at 50 nM. All other capturing and imaging conditions were the same as for the individual miRNA experiments shown in FIG. 13B.

### Example 8—Ratio-Metric Detection of miRNAs Through Kineto-Optical Multiplexing

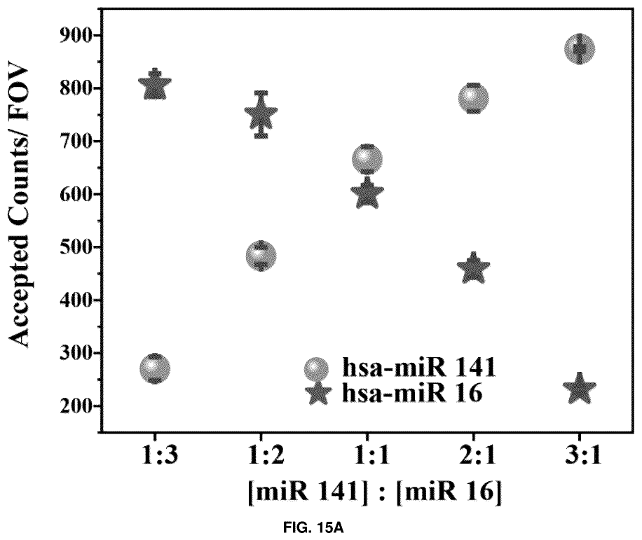

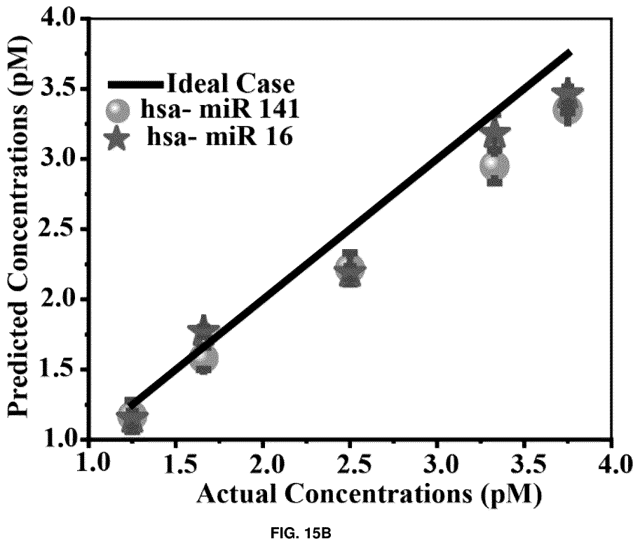

During the development of embodiments of the technology described herein, experiments were conducted to use ratio-metric multiplexing to detect two miRNAs (i.e., hsa-miR 141 and hsa-miR 16). FIG. 15A. hsa-miR 16 was in the cy5 channel and had slow kinetics (i.e., <τₒₙ>=3.10±0.03 s), and hsa-miR 141 was in the cy3 channel and had fast kinetics (i.e., <τₒₙ>=0.28±0.02 s). Capture probes (CPs) were used at 50 nM, and fluorophores (FPs) were used at 50 nM. miRNA targets were mixed in different ratios to provide a total miRNA concentration of 5 pM. All other capturing conditions, imaging conditions, SiMREPS analysis pipelines, and threshold parameters were the same as for the individual miRNA experiments shown in 13B. Data are shown as the mean±SD of n=4 independent measurements.

FIG. 15B shows data for determining the concentration of analytes using accepted counts obtained per FOV from the same multiplexing assay as in FIG. 15A (normalized by the slope of the standard curves of individual miRNAs). Data are presented as the mean±SD of n=4 independent measurements. Predicted concentrations were unaligned with the actual concentrations in the higher concentration range (˜5 pM), which was expected due to the assay being performed close saturation levels.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1-67. (canceled)

68. A multiplex method for detecting a plurality of analytes, said method comprising:
stably binding a first analyte to a solid support;
stably binding a second analyte to the solid support;
providing a first query probe comprising a first detectable label, wherein the first query probe is specific for the first analyte;
providing a second query probe comprising a second detectable label, wherein the second query probe is specific for the second analyte;
recording a first time-dependent change in a first signal intensity of the first detectable label; and
recording a second time-dependent change in a second signal intensity of the second detectable label.

69. The method of claim 68, wherein the first detectable label and the second detectable label are the same.

70. The method of claim 68, wherein the first detectable label and the second detectable label are different.

71. The method of claim 68, wherein the first analyte comprises a nucleic acid, a protein, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism.

72. The method of claim 68, wherein the second analyte comprises a nucleic acid, a protein, a small molecule, a lipid, a carbohydrate, a polysaccharide, a fatty acid, a phospholipid, a glycolipid, a sphingolipid, an organic molecule, an inorganic molecule, a cofactor, a pharmaceutical, a bioactive agent, a cell, a tissue, or an organism.

73. The method of claim 68, comprising using the first time-dependent change in the first signal intensity to characterize, identify, quantify, and/or detect the first analyte and using the second time-dependent change in the second signal intensity to characterize, identify, quantify, and/or detect the second analyte.

74. The method of claim 68, wherein the first time-dependent change in the first signal intensity and the second time-dependent change in the second signal intensity are recorded in a single field of view.

75. The method of claim 68, comprising using Poisson statistical treatment to distinguish the first time-dependent change in the first signal intensity and the second time-dependent change in the second signal intensity.

76. The method of claim 68, wherein the first time-dependent change in the first signal intensity and the second time-dependent change in the second signal intensity are distinguishable by a difference in one or more of minimum values of N_{b+d}, maximum values of N_{b+d}, signal intensity, dwell time in the unbound state, dwell time in the bound state, kinetic dissociation constant, and/or kinetic association constant.

77-95. (canceled)

96. The method of claim 68, wherein a repeated transient association of the first query probe with the first analyte produces the first time-dependent change in the first signal intensity of the first detectable label and a repeated transient association of the second query probe with the second analyte produces the second time-dependent change in the second signal intensity of the second detectable label.

97. The method of claim 68, wherein the first and second analytes remain bound to the solid support during the recording steps.

98. The method of claim 68, wherein the solid support comprises a first immobilized capture probe and a second immobilized capture probe; and stably binding the first analyte to the solid support comprises stably binding the first analyte to the first immobilized capture probe and stably binding the second analyte to the solid support comprises stably binding the second analyte to the second immobilized capture probe.

99. The method of claim 68, wherein the first query probe repeatedly and transiently associates with the first analyte and the second query probe repeatedly and transiently associates with the second analyte during the recording steps.

100. The method of claim 68, further comprising providing a biological sample comprising the first and/or second analyte and contacting the biological sample to the solid support.

101. The method of claim 68, wherein the solid support is diffusible.

102. The method of claim 68, further comprising counting a first number of changes in the signal intensity of the first detectable label and counting a second number of changes in the signal intensity of the second detectable label.

103. The method of claim 68, wherein recording the first time-dependent change in the first signal intensity of the first detectable label and recording the second time-dependent change in the second signal intensity of the second detectable label comprises recording a series of images.

104. The method of claim 103, further comprising producing an intensity fluctuation map by determining an average absolute image-to-image change in intensity at a number of image pixels of the series of images.

105. The method of claim 103, further comprising generating intensity-versus-time data for the first and second analytes and calculating a kinetic parameter from the intensity-versus-time data.

106. The method of claim 105, further comprising identifying positive detection events using a threshold for the kinetic parameter.
